(19) United States
(12) Patent Application Publication
(10) Pub. No.: US 2008/0286349 A1
(43) Pub. Date: Nov. 20, 2008

(54) SYSTEMS, DEVICES, AND METHODS FOR PASSIVE TRANSDERMAL DELIVERY OF ACTIVE AGENTS TO A BIOLOGICAL INTERFACE

(76) Inventors: Youhei Nomoto, Adachi-ku (JP); Kiyoshi Kanamura, Hachioji-shi (JP); Izumi Ishikawa, Machida-shi (JP); Mayuko Ishida, Chofu-shi (JP); Chizuko Ishikawa, Chofu-shi (JP); Akiyoshi Saito, Kawasaki-shi (JP)

Correspondence Address: SEED INTELLECTUAL PROPERTY LAW GROUP PLLC, 701 FIFTH AVE, SUITE 5400, SEATTLE, WA, 98104, US

(21) Appl. No.: 12/122,630

(22) Filed: May 16, 2008

Related U.S. Application Data
(60) Provisional application No. 60/957,126, filed on Aug. 21, 2007.
(60) Provisional application No. 60/956,895, filed on Aug. 20, 2007.
(60) Provisional application No. 60/955,850, filed on Aug. 14, 2007.
(60) Provisional application No. 60/938,961, filed on May 18, 2007.

(51) Int. Cl. A61K 31/473; A61K 9/70; A61K 31/167; A61K 31/196; A61K 31/7048; A61P 11/00

(52) U.S. Cl. 424/449; 514/312; 514/626; 514/570; 514/27

(57) ABSTRACT

Systems, devices, and methods for transdermal delivery of one or more therapeutic active agents to a biological interface. A transdermal drug delivery system is provided for passive transdermal delivery of one or more ionizable active agents to a biological interface of a subject. A transdermal drug delivery system includes a backing substrate, and an active agent layer. The active layer includes a thickening agent, a plasticizer, and a therapeutically effective amount of an ionizable active agent.

### CROSS-REFERENCE AND RELATED APPLICATIONS

This application claims the benefit under 35 U.S.C. § 119(e) of U.S. Provisional Patent Application No. 60/938,961 filed May 18, 2007; U.S. Provisional Patent Application No. 60/955,850 filed Aug. 14, 2007; U.S. Provisional Patent Application No. 60/956,895 filed Aug. 20, 2007; and U.S. Provisional Patent Application No. 60/957,126 filed Aug. 21, 2007.

### BACKGROUND

1. Field of Technology

This disclosure generally relates to the field of topical and transdermal administration of active agents and, more particularly, to systems, devices, and methods for transdermally delivering active agents to a biological interface via passive diffusion.

2. Description of the Related Art

Conventionally administered active agents in the form of, for example, capsules, injectables, ointments, and pills are typically introduced into the body as pulses that usually produce large fluctuations of active agent concentrations in the bloodstream and tissues and, consequently, provide unfavorable patterns of efficacy and toxicity. For example, conventionally administered active agents for obstructive respiratory aliment treatments generally include inhalation aerosols and inhalation solutions typically administered using inhaler devices (e.g., inhalers). Typically, inhaler devices have an active agent, medication, or drug stored in solution, in a pressurized canister, which is attached to a manually actuated pump. To use a standard inhaler device, a user must first exhale, then insert a mouth-piece end of the inhaler device in their mouth, then manually actuate the pump of the inhaler device while retaining the mouth-piece end in their mouth, and then the user may have to hold their breath for a prerequisite amount of time so that the active agent or medication or drug has a chance to be absorbed into the body instead of being exhaled from the user. Some users may find inhaler devices difficult to use. For example, a user of an inhaler device needs the ability to physically manipulate and actuate the inhaler device. Young users or feeble users may have difficulty mustering the coordination necessary to properly use an inhaler device. Additionally, users lacking the ability to hold their breath for the prerequisite time may likewise be unable to take advantage of inhaler devices.

Accordingly, a need exists for providing alternative modes for administering active agents, for example using transdermal delivery devices, to treat obstructive respiratory ailments.

Skin, the largest organ of the human body, offers a painless and compliant interface for systemic drug administration. As compared with injections and oral delivery routes, transdermal drug delivery increases patient compliance, avoids metabolism by the liver, and provides sustained and controlled delivery over long time periods. Transdermal delivery may in some instances, increase the therapeutic value by obviating specific problems associate with an active agent such as, for example, gastrointestinal irritation, low absorption, decomposition due to first-pass effect (or first-pass metabolism or hepatic effect), formation of metabolites that cause side effects, and short half-life necessitating frequent dosing.

Although skin is one of the most extensive and readily accessible organs, it is relatively thick and structurally complex. Thus, it has historically been difficult to deliver certain active agents transdermally. To transport through intact skin into the blood stream or lymph channels, the active agent must penetrate multiple and complex layers of tissues, including the stratum corneum (i.e., the outermost layer of the epidermis), the viable epidermis, the papillary dermis, and the capillary walls. It is generally believed that the stratum corneum, which consists of flattened cells embedded in a matrix of lipids, presents the primary barrier to absorption of topical compositions or transdermally administered drugs.

Due to the lipophilicity of the skin, water-soluble or hydrophilic drugs are expected to diffuse more slowly than lipophilic drugs. While lipid-based permeation enhancers (such as hydrophobic organic substances including vegetable oils) can sometimes improve the rate of diffusion, such permeation enhancers do not mix well with hydrophilic drugs. For example, development of a transdermal vehicle for delivery of Procaterol, a bronchial dilator, has faced numerous difficulties. Procaterol is highly hydrophilic, and delivery through the skin has not been possible when combined with hydrophobic organic substances.

Commercial acceptance of transdermal delivery devices or pharmaceutically acceptable vehicles is dependent on a variety of factors including cost to manufacture, shelf life, stability during storage, efficiency and/or timeliness of active agent delivery, biological capability, and/or disposal issues. Commercial acceptance of transdermal delivery devices or pharmaceutically acceptable vehicles is also dependent on their versatility and ease-of-use.

The present disclosure is directed to overcoming one or more of the shortcomings set forth above, and/or providing further related advantages.

### BRIEF SUMMARY

Transdermal delivery devices and topical formulations are described. In various embodiments, ionizable and ionized active agents can passively permeate through skin to reach the blood stream and ultimately be delivered systemically.

One embodiment describes a passive transdermal delivery device comprising: a backing substrate; and an active agent layer, wherein the active agent layer is substantially anhydrous and oil-free and includes a thickening agent and an ionizable active agent, and wherein the ionizable active agent is electrically neutral in the active agent layer and dissociates into an ionized active agent upon contacting an aqueous medium.

A further embodiment describes a topical formulation comprising: a thickening agent, an ionized active agent; and an aqueous medium, wherein the topical formulation is substantially oil-free.

Yet another embodiment describes a method of treating a condition associated with an obstructive respiratory ailment in a subject comprising: applying to the subject's skin a passive transdermal delivery device comprising: a backing substrate; and an active agent layer, wherein the active agent layer is substantially anhydrous and oil-free and includes a thickening agent and an ionizable active agent, and wherein the ionizable active agent is electrically neutral in the active agent layer and dissociates into an ionized active agent upon contacting an aqueous medium; and allowing the ionizable active agent to dissociate into the ionized active agent.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements, as drawn, are not intended to convey any information regarding the actual shape of the particular elements, and have been solely selected for ease of recognition in the drawings.

FIG. 1 is an isometric view of an active side of a transdermal drug delivery device according to one illustrated embodiment.

FIG. 2A is a plan view of the active side of the transdermal delivery device of FIG. 1 according to one illustrated embodiment.

FIG. 2B is an exploded view of the transdermal delivery device of FIG. 1 according to one illustrated embodiment.

FIG. 3 is an isometric view of a bottom side of an active side of a transdermal delivery device according to one illustrated embodiment.

FIG. 4A is a plan view of the active side of a transdermal delivery device according to one illustrated embodiment.

FIG. 4B is an exploded view a transdermal delivery device according to one illustrated embodiment.

FIG. 5 schematically illustrate ionic flux-induced electrical field.

FIG. 6 shows ion movements over time (Δt).

FIG. 7 schematically shows an H-shaped Franz cell for testing ionic permeations.

FIGS. 8A-8C illustrate how electrical potential differences influence ionic movement.

FIG. 9 shows a relationship between permeability rate of Procaterol cations within the skin and the concentration of Procaterol HCl.

FIG. 10 shows the actual amount of aqueous Procaterol delivered to hairless mouse skin over time measured using the Franz cell of FIG. 7 at a number of different concentrations.

FIG. 11 shows the computed values compared with the actual measured values in FIG. 10.

FIG. 12 shows a relationship between the concentration of sodium Diclofenac and the delivery rate of Diclofenac anions to the skin.

FIG. 13 shows the electric potential difference generated within the skin as a result of ionic diffusion.

FIG. 14 compares the measured results with the computed (predicted) results of FIG. 13.

FIG. 15 shows a relationship between the concentration of AA2G and AA2G− ions within the skin.

FIG. 16 shows the electric potential difference occurring within the skin.

FIG. 17 shows a comparison between the computational results and the experimental results.

FIG. 18 shows a relationship between the concentration of Lidocaine HCl and Lidocaine cations delivered within the skin.

FIG. 19 shows the electric potential difference generated during delivery of Lidocaine HCl within the skin.

FIG. 20 shows a comparison of computed and actual experimental values of Lidocaine HCl permeation.

FIG. 21 is a flow diagram of an exemplary method for manufacturing a transdermal drug delivery device according to one illustrated embodiment.

FIGS. 22A-22C show a spin-coating process according to one illustrated embodiment.

FIG. 23A is a Dynamic Light Scattering measurement plot of Frequency versus Particle Size according to one illustrated embodiment.

FIG. 23B is a cross sectional view of an active agent layer illustrating the interactions of HPC and Procaterol HCl according to one illustrated embodiment.

FIG. 24 is a flow diagram of an exemplary method of preventing or treating a condition associated with an obstructive respiratory ailment according to one illustrated embodiment.

FIG. 25A is an exploded view of a test diffusion cell for evaluating in vitro transdermal permeation according to one illustrated embodiment.

FIGS. 25B and 25C show an exploded and an unexploded view of a Franz test diffusion cell for evaluating in vitro transdermal permeation according to one illustrated embodiment.

FIG. 26 is a plot of Procaterol HCl Delivered versus Time according to one illustrated embodiment.

FIG. 27 is an exemplary permeation profile of Procaterol to a Phosphate Buffered Saline (PBS) versus Time plot according to one illustrated embodiment.

FIG. 28 is a plot of permeation profile of Procaterol to a Phosphate Buffered Saline (PBS) versus Time for an exemplary embodiment of a delivery device.

FIG. 29 is plot of permeation profile of Procaterol to a Phosphate Buffered Saline (PBS) versus Time for an exemplary embodiment of a delivery device.

FIG. 30 is a plot of permeation profile of Procaterol to a Phosphate Buffered Saline (PBS) versus Time for an exemplary embodiment of a delivery device.

FIG. 31 is a plot of permeation profile of Procaterol to a Phosphate Buffered Saline (PBS) versus Time for an exemplary embodiment of a delivery device.

FIG. 32 is a plot of permeation profile of Procaterol to a Phosphate Buffered Saline (PBS) versus Time for an exemplary embodiment of a delivery device.

### DETAILED DESCRIPTION

In the following description, certain specific details are included to provide a thorough understanding of various disclosed embodiments. One skilled in the relevant art, however, will recognize that embodiments may be practiced without one or more of these specific details, or with other methods, components, materials, etc. In other instances, well-known structures associated with delivery devices including, but not limited to, protective coverings and/or liners to protect delivery devices during shipping and storage have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment,” or “an embodiment,” or “in another embodiment,” or “in some embodiments” means that a particular referent feature, structure, or characteristic described in connection with the embodiment is included in at least one embodiment. Thus, the appearance of the phrases “in one embodiment,” or “in an embodiment” or “in another embodiment,” or “in some embodiments” in various places throughout this specification are not necessarily all referring to the same embodiment. Furthermore, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to an active agent includes a single active agent, or two or more active agents. It should also be noted that the term “or” is generally employed in its sense including “and/or” unless the content clearly dictates otherwise.

It is conventionally believed that ionic drugs do not easily permeate through the skin and are generally not suited for topical formulations (e.g., creams and lotions) or transdermal patches. However, according to the various embodiments described herein, certain ionizable active agents are capable of permeating skin and entering into blood stream or lymph channels. Based on both theoretical models and empirical results of ion permeation within the skin, it is described herein a logical approach to designing transdermal delivery devices (e.g., patches) and topical formulations to passively deliver an ionized active agent. Also described are methods of making and using the same.

### Transdermal Delivery Device

One embodiment provides a passive transdermal delivery device, such as a transdermal patch, comprising a backing substrate and an active agent layer, wherein the active agent layer is substantially anhydrous and oil-free and includes a thickening agent and an ionizable active agent, and wherein the ionizable active agent is electrically neutral in the active agent layer and dissociates into an ionized active agent upon contacting an aqueous medium.

As used herein, “transdermal delivery” refers to passive diffusion of ionic active agents in the absence of externally-applied electrical current. However, as a result of diffusion through skin, the ionic substances establish a concentration gradient, which can give rise to an electrical potential difference on either side of the skin. The electrical potential difference may speed up or hamper the ionic diffusion process, depending on a host of interrelating factors, including the velocity, flux and size of the various ions. It is discussed herein that ionic passive diffusion under controlled conditions can benefit from the dual effects of the electrical potential as well as the concentration gradient.

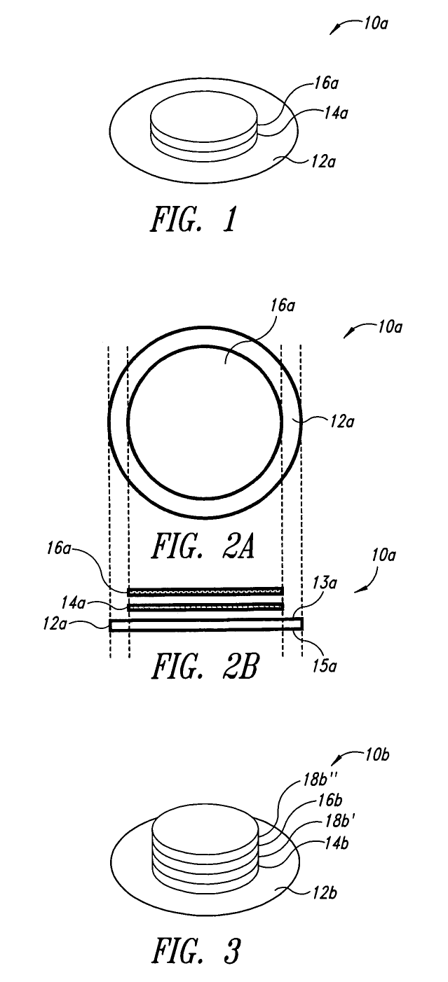

FIGS. 1, 2A, and 2B show a first exemplary embodiment of a delivery device 10a. In some embodiments, the delivery device 10a is configured to transdermally deliver one or more therapeutic active agents to a biological interface of a subject via passive diffusion. As used herein, “biological interface” refers to both skin and mucosal membrane (such as nasal membrane). Unless specified otherwise, all descriptions regarding skin permeation also apply to mucosal membranes. The delivery device 10a includes a backing substrate 12a having opposed sides 13a and 15a. An optional base layer 14a is disposed and/or formed on the side 13a of the backing substrate 12a. An active agent layer 16a is disposed and/or formed on the base layer 14a. The backing substrate 12a, the optional base layer 14a, and the active agent layer 16a may be formed from pliable materials such that the delivery device 10a will conform to the contours of the subject.

FIG. 1 shows an isometric view of the delivery device 10a. When the delivery device 10a is placed on a subject (not shown), the active agent layer 16a is proximal to the subject and the backing substrate 12a is distal to the subject. The backing substrate 12a may include an adhesive such that the delivery device 10a may be applied to the subject and be adhered thereon. In some embodiments, the backing 12a encases the delivery device 10a. Non-limiting examples of backing substrates include 3M™ CoTran™ Backings, 3M™ CoTran™ Nonwoven Backings, and 3M™ Scotchpak™ Backings.

The optional base layer 14a may be constructed out of any suitable material including, for example, polymers, thermoplastic polymer resins (e.g., poly(ethylene terephthalate)), and the like. In some embodiments, the optional base layer 14a and the active agent layer 16a may cover a substantial portion of the backing substrate 12a. For example, in some embodiments, the backing substrate 12a, the optional base layer 14a, and the active agent layer 16a may be disk shaped and the backing substrate 12a may have a diameter of approximately 15 millimeter (mm) and the optional base layer 14a and the active agent layer 16a may have respective diameters of approximately 12 mm. In some embodiments, the sizes of the backing substrate 12a, the base layer 14a, and the active agent layer 16a may be larger or smaller, and in some embodiments, the relative size differences between the backing substrate 12a, the base layer 14a, and the active agent layer 16a may be different from that shown in FIGS. 1, 2A, and 2B. In some embodiments, the size of the active agent layer 16a may depend upon, among other things, the active agent or active agents being delivered by the delivery device 10a and/or the rate at which the active agent or active agents are to be delivered by the delivery device 10a. Typically, the backing substrate 12a and the base layer 14a are sized to the active agent layer 16a such that the sizes of the backing substrate 12a and the base layer 14a are least the size of the active agent layer 16a.

FIG. 3 shows a second embodiment of a delivery device 10b. In this embodiment, the elements and features labeled with a reference numeral and the letter “b” corresponds to features and components that are similar at least in some respects as those of FIGS. 1, 2A, and 2B that are labeled with the same reference numeral and the letter “a”. This embodiment may be effective in enhancing the delivery of an active agent in instances including, but not limited to, where the active agent has unfavorable dissolving kinetics and may also be employed in instances where the dissolving kinetics of the active agent are not unfavorable.

The delivery device 10b includes, a backing substrate 12b, a base layer 14b, and an active agent layer 16b storing one or more ionizable active agents. It has been found that replenishing the ionizable active agent in the active layer 16b may play an important roll for proper delivery of the active agent. In particular, by replenishing the ionizable active agent in the active agent layer 16b (or 16a), it is possible to maintain a concentration of the ionizable active agent in the active agent layer 16b (or 16a) that is fairly or substantially constant over time. Accordingly, in the embodiment illustrated in FIG. 3, the delivery device 10b may include an inner active agent-replenishing layer 18b′ and an outer active agent-replenishing layer 18b″. The active agent-replenishing layers 18b′, 18b″ may be formed from a material (e.g., a thickening agent) such as, but not limited to, hydroxypropyl cellulose (HPC). The active agent-replenishing layers 18b′, 18b″ cache additional ionizable active agents that diffuse into the active agent layer 16b.

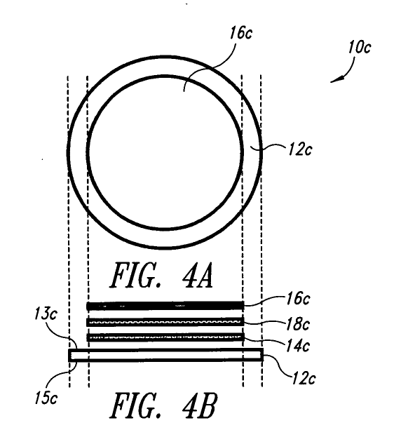

FIGS. 4A and 4B show a third embodiment of a delivery device 10c. In this embodiment, the elements and features labeled with a reference numeral and the letter “c” corresponds to features and components that are similar at least in some respects as those of FIGS. 3A and 3B that are labeled with the same reference numeral and the letter “b”. The delivery device 10c includes an outer active agent-replenishing layer 18c interposing the active agent layer 16c and the base layer 14c. In some embodiments, an active agent-replenishing layer 18c may be disposed on the active agent layer 16c distal from the base layer 14c such that the active agent layer 16c interposes the agent-replenishing layer 18c and the base layer 14c.

In various embodiments, the active agent layer 16a includes a thickening agent and a therapeutically effective amount of an ionizable active agent.

### A. Thickening Agent:

“Thickening agent” refers to an inert and viscous material that provides the bulk of the active agent layer. For example, the thickening agent provides a sol into which the active agent is dispersed. By adjusting the relative amounts of the thickening agent and the active agent, active agent layers of selected concentrations and viscosities can be prepared. Typically, the thickening agent is a cellulose derivative. Exemplary thickening agents include, but are not limited to, polysaccharides (e.g., hydroxypropyl cellulose, hydroxymethyl cellulose, hydroxypropyl methylcellulose and the like) proteins, viscosity enhancers, and the like.

### B. Ionizable Active Agent:

“Active agent” refers to a compound, molecule, or treatment that elicits a biological response from any host, animal, vertebrate, or invertebrate, including, but not limited to, fish, mammals, amphibians, reptiles, birds, and humans. Non-limiting examples of an active agent includes a therapeutic agent, a pharmaceutical agent, a pharmaceutical (e.g., a drug, a therapeutic compound, a pharmaceutical salt, and the like), a non-pharmaceutical (e.g., a cosmetic substance, and the like), a vaccine, an immunological agent, a local or general anesthetic or painkiller, an antigen or a protein or peptide such as insulin, a chemotherapy agent, and an anti-tumor agent.

An ionizable active agent refers to an active agent, as defined herein, that is electrically neutral (i.e., non-ionized) prior to contacting an aqueous medium. Upon contacting an aqueous medium, the ionizable active agent dissociates into an “ionized active agent” and a counterion. Depending on the chemical structure of the ionizable active agent, the ionized active agent can be cationic or anionic. As used herein, an aqueous medium refers to a water-containing environment, including moisture, aqueous solution (e.g., saline solution), and sweat present on skin.

Typically, the ionizable active agent is a salt. In certain embodiments, an active agent containing one or more amines (including primary, secondary and tertiary amine) or imines can be converted into an ionizable salt form in the presence of an acid. Preferably, the active agent has a tertiary amine or secondary amine and the acid is a strong acid such as hydrochloride acid (HCl). The salt dissociates into a cationic active agent (containing a positively-charged ammonium ion) and a counter ion (e.g., chloride). Thus, the acid (organic or inorganic) is selected such that the counter ion is physiologically compatible. Exemplary acids include, for example, phosphoric acid (phosphate counterion), citric acid (citrate counterion), acetic acid (acetate counterion), lactic acid (lactate counterion) and so forth.

Thus, in certain embodiments, the ionizable active agent that produces a cationic active agent is an amine-containing drug. In one embodiment, the active agent layer includes Procaterol as a pharmaceutically acceptable salt, i.e., 8-hydroxy-5-[1-hydroxy-2-[(1-methylethyl)amino]butyl]-2(1H)-quinolinone, [(R*,S*)-(+−)-8-hydroxy-5-(1-hydroxy-2-((1-methylethyl)amino)butyl)-2(1H)-quinolinone] as a pharmaceutically acceptable salt. See, e.g., U.S. Pat. No. 4,026,897 which is hereby incorporated by reference in its entirety. Suitable salt forms of Procaterol include Procaterol HCl and its hydrate forms, including Procaterol HCl hemihydrate, Procaterol HCl hydrate, and respective isomers thereof:

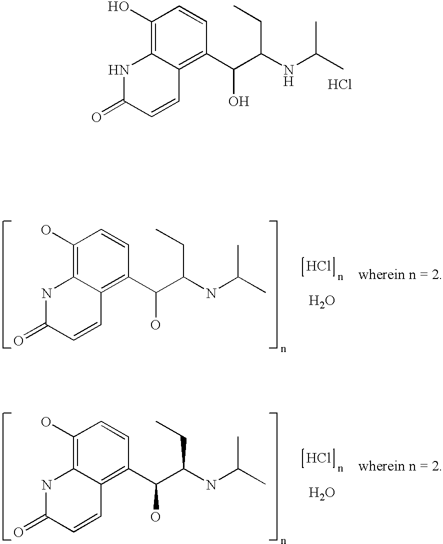

Procaterol is one example of a class of amine-containing β-adrenergic agonists. Other examples of amine-containing β-adrenergic agonists include Arformoterol, Bambuterol, Bitolterol, Clenbuterol, Fenoterol, Formoterol, Hexoprenaline, Isoetarine, Levosalbutamol, Orciprenaline, Pirbuterol, Procaterol, Reproterol, Rimiterol, Salbutamol, Salmeterol, Terbutaline, Tretoquinol, Tulobuterol, and the like.

In further embodiments, the amine-containing ionizable active agent is a “caine”-type analgesic or anesthetic. In particular, the ionizable active agent is a salt form of Lidocaine, e.g., Lidocaine HCl. Other amine-containing “caine” type drugs include, for example, centbucridine, tetracaine, Novocaine® (procaine), ambucaine, amolanone, amylcaine, benoxinate, betoxycaine, carticaine, chloroprocaine, cocaethylene, cyclomethycaine, butethamine, butoxycaine, carticaine, dibucaine, dimethisoquin, dimethocaine, diperodon, dyclonine, ecogonidine, ecognine, euprocin, fenalcomine, formocaine, hexylcaine, hydroxyteteracaine, leucinocaine, levoxadrol, metabutoxycaine, myrtecaine, butamben, bupivicaine, mepivacaine, beta-adrenoceptor antagonists, opioid analgesics, butanilicaine, ethyl aminobenzoate, fomocine, hydroxyprocaine, isobutyl p-aminobenzoate, naepaine, octacaine, orthocaine, oxethazaine, parenthoxycaine, phenacine, piperocaine, polidocanol, pramoxine, prilocaine, propanocaine, proparacaine, propipocaine, pseudococaine, pyrrocaine, salicyl alcohol, parethyoxycaine, piridocaine, risocaine, tolycaine, trimecaine, tetracaine, anticonvulsants, antihistamines, articaine, cocaine, procaine, amethocaine, chloroprocaine, marcaine, chloroprocaine, etidocaine, prilocaine, lignocaine, benzocaine, zolamine, ropivacaine, dibucaine, as pharmaceutically acceptable salt thereof, or mixtures thereof.

In other embodiments, the ionizable active agent contains one or more carboxylic acids (—COOH), which can be in a salt form. This type of ionizable active agent dissociates into anionic active agent and a physiologically compatible counterion. For example, in certain embodiments, the ionizable active agent is an alkaline salt of Diclofenac. Diclofenac is a non-steroidal anti-inflammatory drug (NSAID). The sodium salt of Diclofenac (i.e., monosodium 2-(2-(2,6-dichlorophenylamino)phenyl)acetate) has the following general molecular formula:

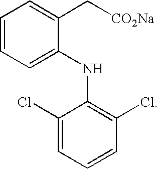

Other suitable physiologically-compatible counterions include, for example, ammonium, potassium and so forth.

In other embodiments, the ionizable active agent is a salt of ascorbic acid or a derivative thereof. Ascorbic acid is an antioxidant and inhibits melanogenesis. Its salt form can dissociate into ascorbate anion and a positively charged counterion. For example, the sodium salt of ascorbic acid (or sodium ascorbate in L or D form) is shown below:

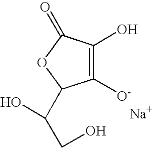

In certain embodiments, the ionizable active agent is a stable ascorbic acid derivative: L-Ascorbic acid 2-Glucoside (AA2G) dissociates into AA2G (−) and a proton.

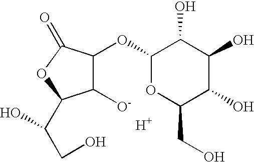

In some instances, once permeate into the skin, ionized active agents can rapidly depart from the lipophilic bilayers in the skin and reach deeper into the tissue, and ultimately reach the blood stream and deliver systemically.

Polarizable active agents are also within the scope of suitable active agents. “Polarizable active agent” is also electrically neutral but exhibits more polarity at one portion relative to another portion in the presence of a polar solvent (such as an aqueous medium, as defined herein).

### C. Optional Components

In addition to the thickening agent and the ionizable active agent, the active agent layer 16a may further include one or more optional components such as an ionizable additive, a humectant, a plasticizer and a permeation enhancer.

“Ionizable additive” refers to an inert salt that produces ions upon contact with an aqueous medium. As discussed in more detail herein, the ionizable additive dissociated ions that contribute to the formation of concentration gradient and influence the electrical potential induced by ion flux during the ionic permeation process. Advantageously, based on their permeation characteristics, suitable ionizable additive can be selected to aid the permeation process of the ionized active agent. Exemplary ionizable additives include potassium chloride (KCl), sodium chloride (NaCl), and the like.

In some embodiments, the active agent layer 16a may include a humectant. Exemplary humectants include, but are not limited to, hygroscopic substances, molecules having several hydrophilic groups (e.g., hydroxyl groups, amines groups, carboxyl groups, esterified carboxyl groups, and the like), compounds having an affinity to form hydrogen bonds with water molecules, and the like. Further examples of humectants include, but are not limited to, urea, glycerine, propylene glycol (E 1520) and glyceryl triacetate (E1518), polyols (e.g., sorbitol (E420), xylitol and maltitol (E965), polymeric polyols (e.g., polydextrose (E1200), natural extracts (e.g., quillaia (E999), and the like.

In some embodiments, the active agent layer 16a may include a plasticizer. The term “plasticizer” or “softener” typically refers to a substance, compound, or mixture that is added to increase the flexibility of the thickening agent. Suitable plasticizers include polyglycols polyglycerols, polyols, polyethylene glycols (PEG, polyethylene glycols (e.g., PEG-200, PEG-300, PEG-400, PEG-4000, PEG-6000), di(2-ethylhexyl)phthalate (DEHP), triethylene glycol, and the like.

In some embodiments, combining a one or more organic components with an active agent may promote or enhance absorption of the active agent into the skin. For example, surfactants may alter protein structure or fluidize skin and increase permeation. In some embodiments, absorption of ionic or polar active agents may be enhanced by including surfactants with hydrophilic head groups. A lipophilic portion of the surfactant may assist the permeation through skin.

Optionally, the active agent layer may include additional agents such as analgesics, anesthetics, anesthetics vaccines, antibiotics, adjuvants, immunological adjuvants, immunogens, tolerogens, allergens, toll-like receptor agonists, toll-like receptor antagonists, immuno-adjuvants, immuno-modulators, immuno-response agents, immuno-stimulators, specific immuno-stimulators, non-specific immuno-stimulators, and immuno-suppressants, or combinations thereof.

### D. Dosage and Formulation of the Active Agent Layer

In certain embodiments, the active agent layer is substantially anhydrous and oil-free. It is considered “substantially anhydrous” when the active agent layer contains no more than 5% by weight of water, and more typically, no more than 3%, 2%, 1% or 0.5% of water. Under the substantially anhydrous condition, the ionizable active agent remains electrical neutral, which is generally more stable than its ionized form. Thus, longer shelf-life of the active agent can be expected. It is consider “substantially oil-free” when the active agent layer contains no more than 5% by weight of a lipophilic component such as fatty acids, vegetable oil, petroleum or mineral oil, including short chain (e.g., fewer than 14 carbons) saturated hydrocarbons, silicone oils and the like. These conventional permeation enhancers are not necessary to provide assistance to ionic permeation. On the other hand, because oil tends to destabilize the ionizable or ionized active agent during storage or delivery, an oil-free active agent layer is expected to provide long-term stability to the active agent.

In various embodiments, the amount of ionizable active agent in the active agent layer depends on both its permeation rate and dosage regimen. In addition, the concentration of the ionizable active agent in the active agent layer 16a is selected dependent on factors such as, but not limited to, the solubility of the ionized active agent, the rate of solution of the ionizable active agent, and so forth.

The initial loading of the ionizable active agent also influences the permeation of the ionized active agent. Higher concentration of the ionizable active agent can lead to higher permeation rate. Thus, it is desirable to load maximum amount of the active agent within a minimum amount of the thickening agent (i.e., forming the highest concentration of active agent in a thinnest active agent layer). On the other hand, because the active agent is not typically fully absorbed by the skin, care should be taken to limit the initial loading level to ensure that even at a full dose, the patch is not lethal if ingested. For example, a Procaterol HCl patch typically contains about 25 μg to maximally 100 μg Procaterol HCl.

Typically, the active agent layer may include from about 0.001 wt % to about 10 wt % of an ionizable active agent, more typically, the active agent layer may include from about 0.01 wt % to 5 wt %, or from about 0.01 wt % to 0.1 wt %, 0.1 wt % to 1 wt %, 0.1 wt % to 5 wt % of the an ionizable active agent.

In certain embodiments, the active agent layer comprises HPC and Procaterol HCl. In a more specific embodiment, the active agent layer comprises HPC, Procaterol HCl and urea. In other embodiments, the active agent layer comprises HPC, Procaterol HCl, and glycerol. In other embodiments, the active agent layer comprises HPC, Lidocaine HCl, and glycerol. In other embodiments, the active agent layer comprises HPC and Sodium Diclofenc. In other embodiments, the active agent layer comprises HPC and AA2-G.

In other embodiments, the active agent layer consists essentially of a thickening agent, an ionizable active agent and a humectant. In a particular embodiment, the active agent layer consists essentially of HPC, Procaterol HCl and urea.

### E. Theoretical Model and Empirical Results of Ion Permeation

As discussed, a variety of ionizable active agents are capable of dissociating into ions that transport through the skin. When analyzing the transdermal delivery of an ionic substance into the skin, simple diffusion based upon a concentration gradient cannot provide a complete picture of the events that take place. Without being bound by the following theories, an analysis is provided herein to explain the ionic transdermal mechanism based on electric potential in addition to concentration gradients. It is believed that the driving force for ion transport through a membrane (e.g., skin) relates to both concentration gradients and electric potential gradients induced by the ionic flux. As used herein, “flux” or “ionic flux” refers to the rate of an ionic substance (i.e., ionized active agent) that moves across a unit area. Typically, ionic flux is represented by, e.g., μg cm⁻²·h⁻¹ or mol cm⁻²·h⁻¹.

Eq. 1 describes a basic ion flux J:

$$J = - ϖ cRT ( \frac{d ln c}{d x} + \frac{zF}{RT} \frac{d φ}{d x} ) \\ J = - uc ( \frac{RT}{zF} \frac{d ln c}{d x} + \frac{d φ}{d x} ) \qquad Eq . 1$$

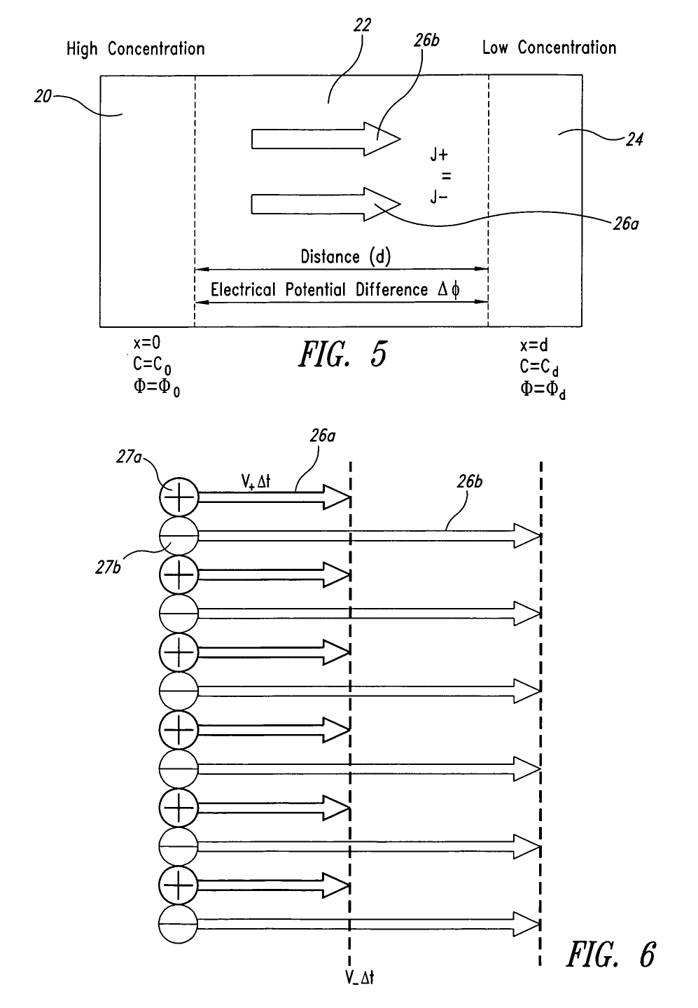

The first term in Eq. 1, often used in analyzing electrochemical systems, relates to ion diffusion while the second term relates to ion movement due to an electric field. FIG. 5 schematically illustrate ionic flux-induced electrical field. As shown, a high concentration of ionic drug solution is placed in the left side chamber 20. A porous membrane 22 corresponding to the surface of the skin is connected to the chamber 20, and the ionic drug solution contacts the porous membrane at a position x=0. The initial concentration of the drug solution is C₀. The thickness of the porous membrane is d, and the concentration of the ionic drug in the chamber 24 to the right of the porous membrane is taken as C_{d}. Diffusion proceeds from the left side chamber 20 toward the right side of the system in FIG. 5, and establishes a concentration gradient, which induces an electrical potential difference.

When cations and anions move through the skin, their velocities are defined in Eq. 2.

$${v}_{+} = - {ϖ}_{+} RT ln \frac{d ln c}{d x} \\ {v}_{-} = - {ϖ}_{-} RT ln \frac{d ln c}{d x} \qquad Eq . 2$$

In Eq. 2, ω₊ and ω₋ represent the molar mobility of cations and anions, respectively, in solution. Cations and anions move independently in solution and in the membrane, but both move according to the same concentration gradient. The relative speeds of anions and cations thus depend only upon Eq. 2. Chemical compounds employed as drugs or cosmetics are often chloride or alkali metal salts of organic substances, meaning that once dissociated into ions, one ion (generally the active agent ion) is much larger than the other. Consequently, the overall size of the drug ion does not change significantly after dissociation, and it is reasonable to expect that the transdermal delivery of the ionic drug due to diffusion (based on concentration gradient) should not differ significantly from that of the neutral molecule.

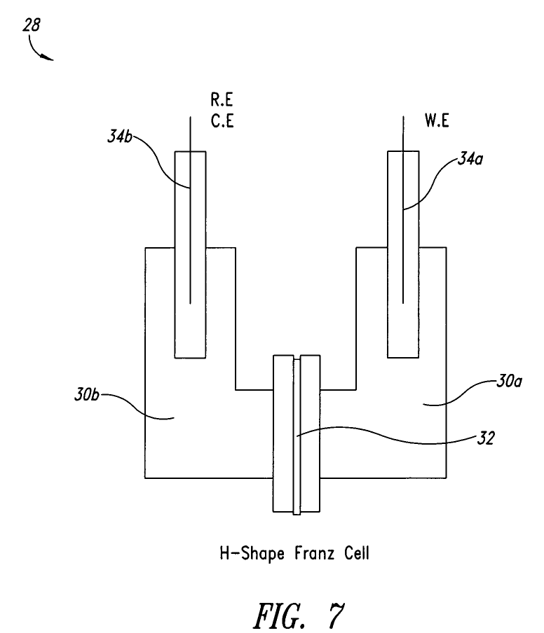

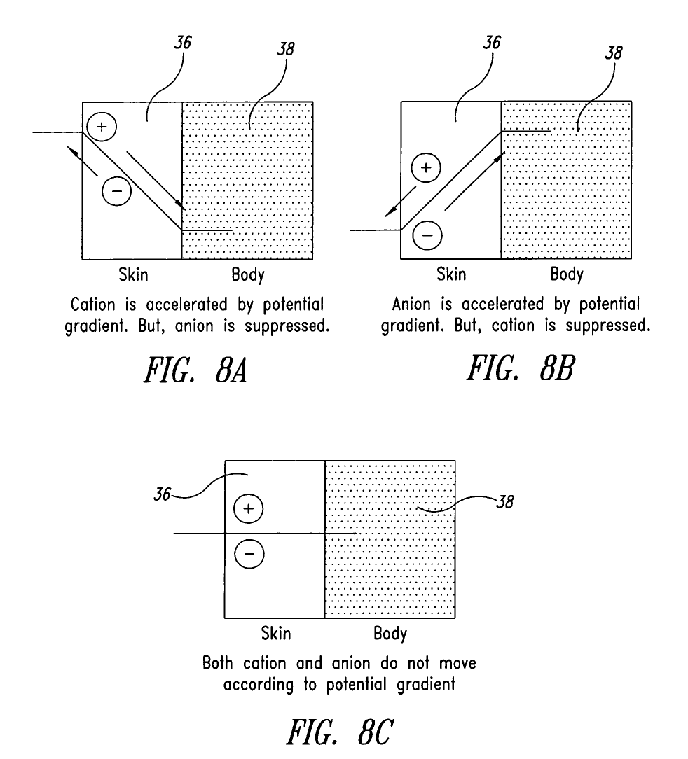

FIG. 6 shows ion movements over time (Δt) when the cation velocity is assumed to be half of that of anions. Cations (27a) move for v+Δt (26a) while anions (27b) move v−Δt (26b). A charge separated state is thus generated in the membrane, leading to an electric potential difference over a very short distance. This electric potential difference will help accelerate cation movement, while slowing anion movement. Eq. 3 describes this effect, which is not seen in the movement of neutral molecules, mathematically. In Eq. 3, Anions and cations move in opposite directions as shown in Eq. 3. Cations are represented using +, while anions are represented using −. Over time both anions and cations move from one side of the membrane to the other, maintaining electro-neutrality.

$${J}_{+} = - {ϖ}_{+} {c}_{+} ( RT ln \frac{d ln {c}_{+}}{d x} + F \frac{d φ}{d x} ) \\ {J}_{-} = - {ϖ}_{-} {c}_{-} ( RT ln \frac{d ln {c}_{-}}{d x} - F \frac{d φ}{d x} ) \qquad Eq . 3$$

Eq. 4 shows the relationship between the velocity (v) and flux (J) of the ions. The concentration of the ions (c₊ and c₋) are identical if the drug being examined consists of monovalent cations and anions, and the velocity of the cation should be the same as that of the anion.

$${J}_{+} = {c}_{+} {v}_{+} \\ {J}_{-} = {c}_{-} {v}_{-} \\ {v}_{+} = - {ϖ}_{+} ( RT ln \frac{d ln {c}_{+}}{d x} + F \frac{d φ}{d x} ) \\ {v}_{-} = - {ϖ}_{-} ( RT ln \frac{d ln {c}_{-}}{d x} - F \frac{d φ}{d x} ) \qquad Eq . 4$$

Accordingly, Eq. 5 must be satisfied:

$${ϖ}_{+} ( RT \frac{d ln c}{d x} + F \frac{d φ}{d x} ) = {ϖ}_{-} ( RT \frac{d ln c}{d x} - F \frac{d φ}{d x} ) \\ ( {ϖ}_{+} - {ϖ}_{-} ) RT \frac{d ln c}{d x} = - ( {ϖ}_{+} + {ϖ}_{-} ) F \frac{d φ}{d x} \\ \frac{d φ}{d x} = - \frac{{ϖ}_{+} - {ϖ}_{-}}{{ϖ}_{+} + {ϖ}_{-}} \frac{RT}{F} \frac{d ln c}{d x} \qquad Eq . 5$$

A relationship between the concentration gradient and the electric potential gradient is thus obtained. Integrating Eq. 5 from 0 to d and from c₀ to c_{d} leads to an expression showing the electric potential difference (Δφ=dφ/dx) through the membrane.

Thus, substituting Eq. 6 in Eq. 3 gives Eq. 7:

$$\begin{matrix}Δ φ = - \frac{{ϖ}_{+} - {ϖ}_{-}}{{ϖ}_{+} + {ϖ}_{-}} \frac{RT}{F} ln \frac{{c}_{d}}{{c}_{0}} \\ \begin{matrix}{J}_{+} = - {ϖ}_{+} c [ RT \frac{d ln c}{d x} + F ( - \frac{{ϖ}_{+} - {ϖ}_{-}}{{ϖ}_{+} + {ϖ}_{-}} \frac{RT}{F} \frac{d ln c}{d x} ) ] \\ = - {ϖ}_{+} c [ ( 1 - \frac{{ϖ}_{+} - {ϖ}_{-}}{{ϖ}_{+} + {ϖ}_{-}} ) \frac{RT}{c} \frac{d c}{d x} ] \\ = - \frac{2 {ϖ}_{+} {ϖ}_{-}}{{ϖ}_{+} + {ϖ}_{-}} RT \frac{d c}{d x}\end{matrix} & Eq . 6 \\ \begin{matrix}{J}_{-} = - {ϖ}_{+} c [ RT \frac{d ln c}{d x} - F ( - \frac{{ϖ}_{+} - {ϖ}_{-}}{{ϖ}_{+} + {ϖ}_{-}} \frac{RT}{F} \frac{d ln c}{d x} ) ] \\ - {ϖ}_{+} c [ ( 1 + \frac{{ϖ}_{+} - {ϖ}_{-}}{{ϖ}_{+} + {ϖ}_{-}} ) \frac{RT}{c} \frac{d c}{d x} ] \\ - \frac{2 {ϖ}_{+} {ϖ}_{-}}{{ϖ}_{+} + {ϖ}_{-}} RT \frac{d c}{d x}\end{matrix} \\ {v}_{+} = \frac{{J}_{+}}{c} = \frac{{J}_{-}}{c} = {v}_{-} = - \frac{2 {ϖ}_{+} {ϖ}_{-}}{{ϖ}_{+} + {ϖ}_{-}} & Eq . 7\end{matrix}$$

At steady state, it is believed that the ion flux is given by the same equation for anions and cations. Diffusion of both ions occurs depending upon the concentration gradient when a drug permeates as dissociated ions, represented by dc/dx and the diffusion coefficient of Eq. 8.

$$D = \frac{2 {ϖ}_{+} {ϖ}_{-}}{{ϖ}_{+} + {ϖ}_{-}} RT \qquad Eq . 8$$

Further, it is necessary to linearly approximate the concentration gradient or the electric potential gradient to solve equation 9. This leads to equation 10. Integrate equation 10 over C₀ to C_{d} after values for x, 0 to d, and c are obtained. This solves for the flux J, as shown in Eq. 11, which is the so-called Goldman equation.

$$\begin{matrix}J = - ϖ RT \frac{d c}{d x} - zF ϖ c \frac{d φ}{d x} & Eq . 9 \\ \frac{d φ}{d x} ( = - E ) = \frac{Δ φ}{d} = \frac{{φ}_{d} - {φ}_{0}}{d} = const \\ thus \\ J = ω RT \frac{d c}{d x} - zF ω c \frac{Δ φ}{d} & Eq . 10 \\ J = \frac{zF ω Δ φ}{d} \frac{{c}_{d} - {c}_{0} exp ( - \frac{zF}{RT} Δ φ )}{exp ( - \frac{zF}{RT} Δ φ ) - 1} & Eq . 11\end{matrix}$$

The potential difference across the skin has been considered for a single component system. In practice, a variety of ionic compounds may be present (including, for example, ionized active agent and ionized additive). Eq. 12 shows a relationship used for multi-component systems.

$${\sum }_{j} {ω}_{j}^{+} {c}_{j , d} + {\sum }_{k} {ω}_{k}^{-} {c}_{k , 0} = ( {\sum }_{j} {ω}_{j}^{+} {c}_{j , 0} + {\sum }_{k} {ω}_{k}^{-} {c}_{k , d} ) exp ( - \frac{F Δ φ}{RT} ) \\ {φ}_{d} - {φ}_{0} = Δ φ = - \frac{RT}{F} ln \frac{{\sum }_{j} {ω}_{j}^{+} {c}_{j , d} + {\sum }_{k} {ω}_{k}^{-} {c}_{k , 0}}{{\sum }_{j} {ω}_{j}^{+} {c}_{j , 0} + {\sum }_{k} {ω}_{k}^{-} {c}_{k , d}} \qquad Eq . 12$$

Thus, a film potential can be calculated provided that the ion mobility (omega) and concentration (c) within the skin are known. The ion transport speed can then be found from the calculated film potential.

As shown above, movement of ions across or within the skin cannot be viewed in a simple diffusion model because the generation of a membrane potential further influences the concentration gradient. It is therefore necessary to experimentally evaluate this phenomenon and effectively use the results in drug product development. It is also desirable to evaluate potential additives based on this theory.

An H-shaped Franz cell (FIG. 7) was used to evaluate the theory described herein. As shown, the Franz cell 28 includes a donor chamber 30a and a receiver chamber 30b. The donor chamber 30a contains an ionic active agent, which permeates through a membrane 32 to reach the receiver chamber 30b. A working electrode 34a was inserted in the donor chamber 30a, while a counter electrode 34b (i.e., reference electrode) was inserted in the receptor chamber 30b. It is possible to measure the electrical potential difference induced by the ionic diffusion/permeation and the concentration gradient thus established.

FIGS. 8A-8C illustrate how electrical potential differences influence ionic movement. As shown, depending on the charges of the ionic active agent (cationic or anionic), its movement can be affected by the electrically potential difference established across the skin. FIGS. 8A-8C further illustrate that, by selecting certain ionizable additive with known permeation characteristics, it is possible to further accelerate the ionic permeation or at least ameliorate an unfavorable condition by canceling out the electrical potential that retards the movement of the ionic drug.

FIG. 8A shows that an electrical potential difference is generated on either side of the skin 36. When the electrical potential is lower on the inside of the skin (contacting the body 38), cation movement is accelerated by the potential difference, while the anion movement is suppressed. Thus, for cationic active agent, it is desirable that a large membrane potential be generated by an ionized additive. For example, an additive dissociates into easily permeable anions and difficult to permeate cations is preferable.

FIG. 8B shows that an electrical potential difference is generated that favors the anion movement while suppressing the cation movement. Thus, if a cationic active agent is to be delivered, it is preferable that an ionized additive is present to cancel out the potential difference that slows down the cation movement.

FIG. 8C shows that no electrical potential difference is generated. Thus, it is preferable to include an ionized additive that dissociates into easily permeable cations and difficult to permeate anions so as to create an electrical potential difference that favors cation movement.

For anionic active agent, the impacts of the electrical potential difference should be reversed as those of the cationic active agent. Thus, in the condition described in FIG. 8A, an additive that dissociates into easily permeable cations and difficult to permeate anions is preferable. In FIG. 8B, it is preferable that the ionized additive cancels out the generated potential difference. For example, an effective additive will have similar permeation speeds within the skin between its dissociated cations and anions. For FIG. 8C, an additive that dissociates into easily permeable anions and difficult to permeate anions is preferable.

As shown, the mobility of ions within the skin can be influenced by the components (e.g., ionizable additive) contained in the drug product if those components also permeate into the skin. Enhancers used in the conventional patches can be used to improve the speed of the drug ions as long as the enhancers are not adversely influenced by the electric potential difference. Therefore, enhancers can be effective when used with the products described herein. Further, changes in the flux due to the drug concentration can also be evaluated. The activity coefficient and the osmotic pressure changes depending upon the drug concentration, and this greatly influence the speed of the ionic drug movement.

In addition to creating ions in the aqueous medium, it is also possible to create ionic dissociation in polar matrixes and solvents. For example, emulsion matrixes where water and oil are mixed using a surfactant may also be applied, as well as a variety of polymers having ether or ester bonding, and organic solvents and mixed organic and water solvents having a dielectric constant of 20 or greater.

Specific ionizable active agents are described in more detail below. As shown, these ionizable active agents can be delivered transdermally in an ionized form (upon dissociation in an aqueous medium). In certain embodiments, the transdermal delivery can be assisted in the presence of an ionizable additive.

1. Procaterol HCl

Potentially adverse side effects may occur if more than 100 μg of Procaterol is placed in a transdermal patch and that patch is mistakenly ingested by a user or other individual. Also, medicinal efficacy and safety considerations make it desirable that Procaterol be delivered at a substantially constant rate. Development relating to transdermal delivery patches using Procaterol HCl has been undertaken in the past, but a patch has not yet been developed by others that is able to optimize both factors, including the amount of drug in patch, and rate of delivery. Thus, in various embodiments, the transdermal delivery device comprises Procaterol HCl in the active agent layer, wherein at least 50%, or at least 60%, or at least 75% or at least 90% of an initial amount (loading) of Procaterol HCl is delivered over a period of 24 hours. Typically, for safety concerns, the remaining Procaterol HCl after delivery should not exceed 50% of the initial loading of Procaterol HCl.

To load Procaterol HCl on a transdermal delivery device (e.g., a patch), an aqueous solution of Procaterol, or more preferably, a viscous sol using hydroxypropyl cellulose (HPC) can be applied on top of a polyethylene terephthalate (PET) film. Typically, no more than 100 micrograms of Procaterol can be loaded. The patch can be dried to remove any water present during the loading process.

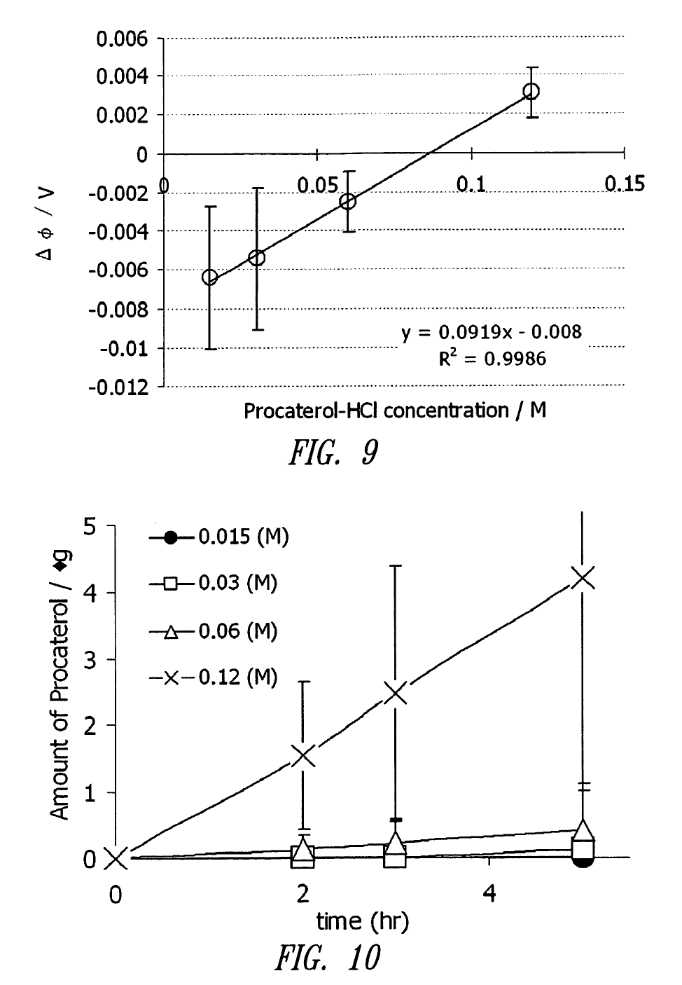

FIG. 9 shows a relationship between permeability rate of Procaterol cations within the skin and the concentration of Procaterol HCl. FIG. 9 also shows the electric potential difference occurring within the skin (measured by using the cell shown in FIG. 7). The electric potential difference shown here is between the outside and the inside of the skin. The “+/−” notation is opposite to that shown in Equation 12. FIG. 9 shows results of measurements made using an aqueous solution of Procaterol HCl. It is thought that the membrane potential may be generated due to the influence of pH changes in solution. At 0.12 M, an electric potential difference is present that tends to promote migration of cations in the direction from the outside of the skin toward the inside due to an electric field within the skin. At other concentrations, however, an oppositely signed electric potential gradient occurs, which would tend to hamper the movement of Procaterol cations into the skin. It is thought that the electric potential differences develop due to the influence of protons, Procaterol cations, and chloride ions.

The mobility of the Procaterol cations with respect to the mobility of chloride ions can be obtained based on the results of the membrane electrical potential measurement. It is noted that the mobilities of Na⁺ and Cl⁻ are nearly the same, as seen from results of measuring the membrane potential using sodium chloride. Also, The mobility of H⁺ is on the order of 1500 times higher than the mobility of Cl⁻, based on the results of measuring the membrane potential using HCl. These values have been used to make calculations. Table 1 shows the results when using 0.12 M Procaterol HCl. Employing values from the Table 1 that are the same as those measured, the ion mobility of Procaterol ions becomes 0.13 with respect to that of chloride ions. It can be seen that the migration speed of Procaterol ions is slow compared to that of chloride ions.

TABLE 1
Concentration of Procaterol: 0.12 M

| | |
|---|---|
| | Concentration |
| Concentration Donor/mol dm⁻³ | Reciever/mol dm⁻³ |

| Cation 1 | Cation 2 | Anion 1 | Cation 1 | Anion 1 |
|---|---|---|---|---|
| Pro+ | H+ | Cl− | Na+ | Cl− |
| 0.12 | 5.01187E−05 | 0.12005 | 0.15 | 0.15 |

| | |
|---|---|
| Mobility of ion | Mobility of ion |

| Cation 1 | Cation 2 | Anion 1 | Cation 1 | Anion 1 |
|---|---|---|---|---|
| 0.13 | 1500 | 1 | 1 | 1 |

| (calc.)/V | (measured)/V |
|---|---|
| −0.00299 | −0.003 V |

In addition, the flux of Procaterol cations can be computed using these results. Results of the calculations made using Eq. 11 are shown in Table 2.

TABLE 2
Concentration of Procaterol: 0.12 M

| Membrane | | | | |
|---|---|---|---|---|
| Potential | Flux (mol) | Mobility ratio | Charge | Faraday Constant |
| /V | J/mol h⁻¹ cm⁻² | w/— | z | F |
| −0.003 | 6.21629E−13 | 0.13 | 1 | 96500 |
| 0.0025 | 2.79666E−13 | 0.13 | 1 | 96500 |
| 0.0054 | 1.32062E−13 | 0.13 | 1 | 96500 |
| 0.0064 | 6.4726E−14 | 0.13 | 1 | 96500 |

| Thickness | | | |
|---|---|---|---|
| of Skin | Concentration/mol cm⁻³ | Mobility of Cl⁻ | Flux |
| d | c | w (Cl⁻) | J/gh⁻¹ cm⁻² |
| 0.01 | 0.00012 | 1.5E−13 | 0.69821339 |
| 0.01 | 0.00006 | 1.5E−13 | 0.314120865 |
| 0.01 | 0.00003 | 1.5E−13 | 0.148331544 |
| 0.01 | 0.000015 | 1.5E−13 | 0.072700284 |

Further, Table 3 shows experimental results of measurements made using a Franz cell. Skin thickness and chloride ion mobility are necessary to apply Eq. 11, and the chloride ion mobility was assumed to be 1.5×10⁻¹³, and the skin thickness was assumed to be 0.01 cm here. The mobility of the chloride ion is on the order of 1/10,000 of that found in an aqueous solution. However, this assumption is thought to be reasonable considering the results for solid polymer electrolytes.

TABLE 3

| | time (hr) | | | | |
|---|---|---|---|---|---|
| concentration | 0 | 2 | 3 | 5 | Flux/mg h⁻¹ cm⁻² |
| 0.015 (M) | 0 | 0 | 0 | 0 | 0 |
| 0.03 (M) | 0 | 0 | 0 | 0.12 | 0.024 |
| 0.06 (M) | 0 | 0.13 | 0.22 | 0.41 | 0.082 |
| 0.12 (M) | 0 | 1.53 | 2.47 | 4.20 | 0.84 |

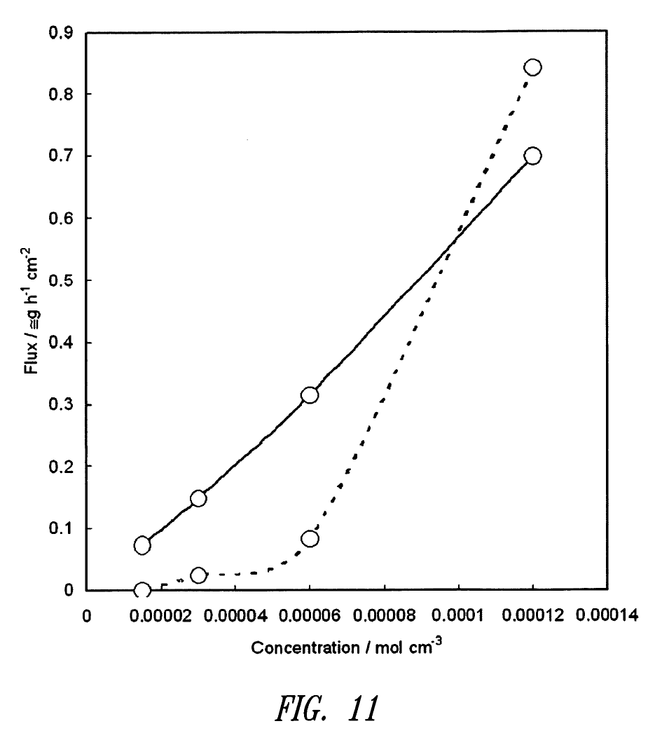

Table 3 shows the actual amount of aqueous Procaterol delivered to hairless mouse skin over time was measured using the Franz cell of FIG. 7 at a number of different concentrations (FIG. 10), as measured delivery rates. The computed values are compared with the actual measured values in FIG. 11. The trend between the two has good agreement, and it would appear that flux values can be reliably predicted using Eq. 11 independently of any actual experimental measurements.

2. Sodium Diclofenac

High concentrations of sodium Diclofenac do not easily dissolve in water, and it is thus customary to use a hydrophobic solvent. However, many hydrophobic solvents are irritating to the skin, and therefore cannot readily be used for a patch medication.

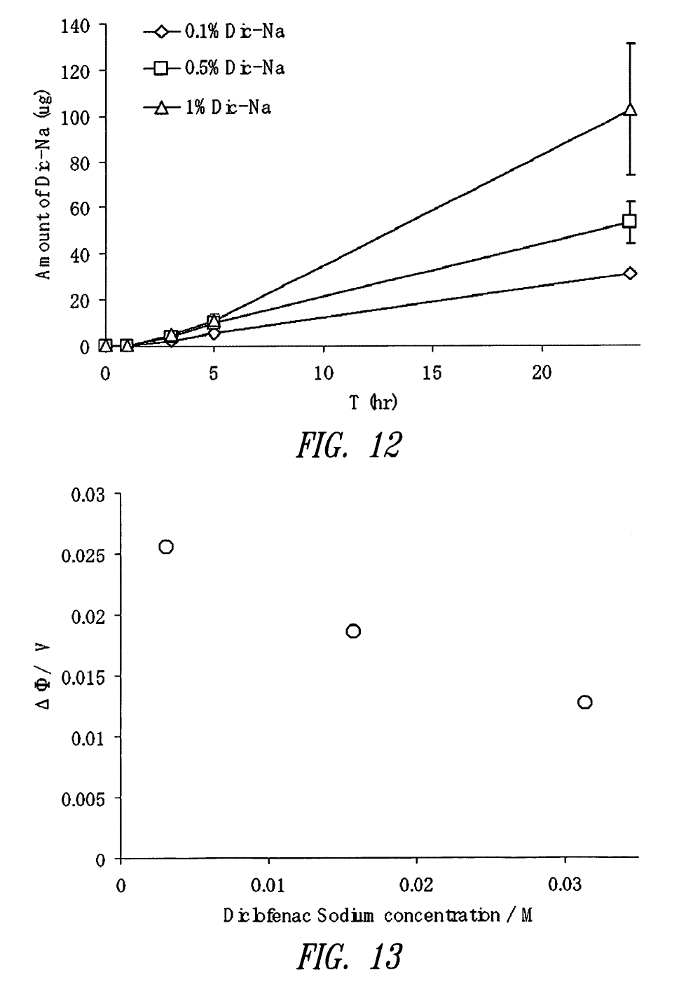

In certain embodiments, a transdermal delivery device including sodium Diclofenac and an ionizable additive is capable of delivering therapeutically effective amount of Diclofenac in an aqueous condition (e.g., upon contacting skin and sweat on the skin). Sodium Diclofenac dissociates into Diclofenac anions and sodium cations. The mobility of Diclofenac anions was found by performing measurements of the membrane potential of the skin. FIG. 12 shows a relationship between the concentration of sodium Diclofenac and the delivery rate of Diclofenac anions (diC⁻) to the skin. FIG. 13 shows the electric potential difference generated within the skin. Results shown in Table 4 are obtained for the mobility based on the data shown in FIGS. 12 and 13.

TABLE 4

| | |
|---|---|
| Concentration | Concentration |
| Donor/mol dm⁻³ | Receiver/mol dm⁻³ |

| Cation 1 | Anion 1 | Cation 1 | Anion 1 |
|---|---|---|---|
| Na+ | diC⁻ | Na+ | Cl⁻ |
| 0.032 | 0.032 | 0.15 | 0.15 |

| | |
|---|---|
| Mobility of ion | Mobility of ion |

| Cation 1 | Anion 1 | Cation 1 | Anion 1 |
|---|---|---|---|
| 1 | 4.6 | 1 | 1 |

| Calculated | Measured |
|---|---|
| −0.012778645 | −0.0127 V |

The mobility of Diclofenac anions was found to be 4.6 (compared to that of chloride ions). This means that Diclofenac anions can be more easily delivered to the skin than chloride ions. Further, computational results shown in Table 5 can be obtained for the Diclofenac flux.

TABLE 5

| | | | | | Thickness | | Mobility | |
|---|---|---|---|---|---|---|---|---|
| Membrane | Flux | Mobility | | Faraday | of | Concentration/ | of | |
| Potential | J/mols⁻¹ | ratio | Charge | Constant | Skin | mol cm⁻³ | Cl⁻ | Flux |
| Δφ/V | cm⁻² | w | z | F | d | c | wC1 | J/μg h⁻¹ cm⁻² |
| −0.01268323 | 4.29808E−12 | 4.6 | −1 | 96500 | 0.01 | 0.000032 | 1.5E−13 | 4.938231541 |
| −0.018592338 | 1.8966E−12 | 4.6 | −1 | 96500 | 0.01 | 0.000016 | 1.5E−13 | 2.179079837 |
| −0.025603065 | 3.25282E−13 | 4.6 | −1 | 96500 | 0.01 | 0.0000032 | 1.5E−13 | 037372945 |
| −0.025079581 | 1.64551E−13 | 4.6 | −1 | 96500 | 0.01 | 0.0000016 | 1.5E−13 | 0.189059535 |
| −0.021835361 | 3.53546E−14 | 4.6 | −1 | 96500 | 0.01 | 0.0000032 | 1.5E−13 | 0.040620334 |

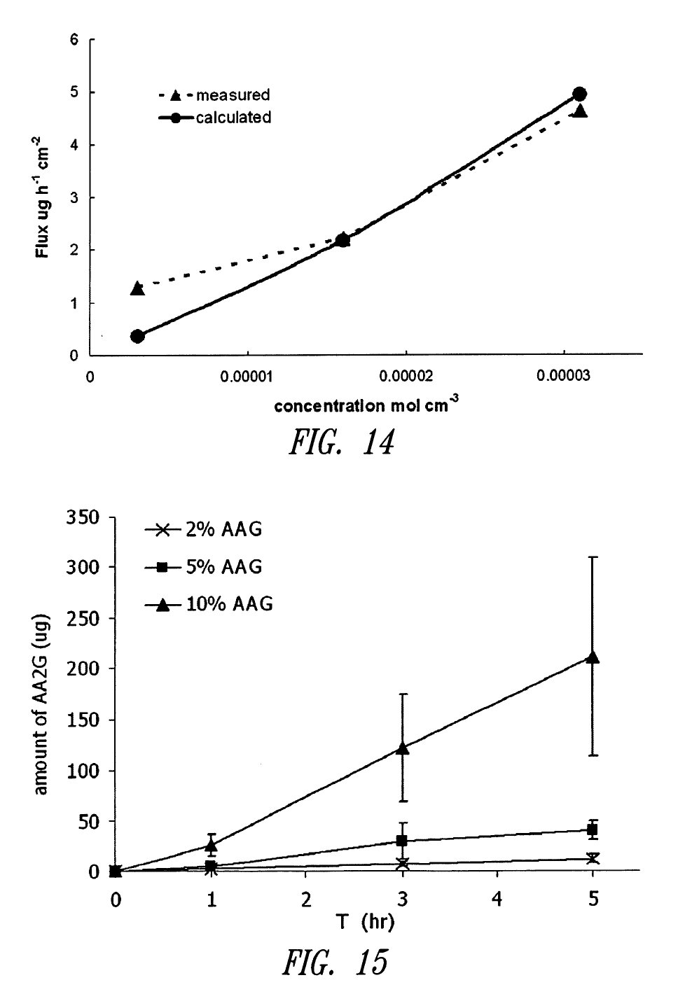

Table 6 shows measured results. FIG. 14 compares the measured results with the computed (predicted) results. A correlation can be seen between the computational results and the actual measured values. It is thus possible to predict the delivery rate of Diclofenac ions using the mobility obtained from measurement of the membrane potential.

TABLE 6

| | time (hr) | | | | | Flux |
|---|---|---|---|---|---|---|
| Concentration/M | 0 | 1 | 3 | 5 | 24 | μg h⁻¹ cm⁻² |
| 0.003 | 0.0 | 0.0 | 2.0 | 5.6 | 31.1 | 1.30 |
| 0.016 | 0.0 | 0.0 | 3.6 | 9.8 | 53.1 | 2.21 |
| 0.031 | 0.0 | 0.3 | 5.4 | 12.1 | 110.9 | 4.62 |

The membrane potential shows negative values. Anions thus pass into the skin while undergoing a deceleration. It follows that, by reducing the potential difference occurring within the skin to zero, or making it positive, it is possible to improve the delivery rate. One possible method considered is to use KCl as an additive. KCl dissociates into K⁺ and Cl⁻ ions. From separate membrane potential measurements, the mobility of K⁺ within the skin was found to be large compared to that of Cl⁻. It is thus thought that KCl could be used to lower the negative electric potential gradient occurring within the skin. 0.1% and 0.5% KCl was added to the Diclofenac solution and measurements of membrane potential were performed, the results of which are shown in Table 7.

TABLE 7
1.0% Diclofenac

| | | experimental | calculated |
|---|---|---|---|
| | | Flux | Flux |
| KCl conc (%) | ΔΦ V | J/μg h⁻¹ cm⁻² | J/μg h⁻¹ cm⁻² |
| 0 | −0.013 | 4.3 | 4.8 |
| 0.1 | −0.0075 | 6.2 | 5.4 |
| 0.5 | 0.002 | 7.0 | 6.5 |

The membrane potential difference indeed became smaller upon addition of the KCl additive, which reduced the electric potential gradient that tends to hinder delivery of Diclofenac into the skin. It can be seen that the amount that the electric potential gradient is reduced depends upon the amount of KCl added. In addition, it can also be seen that a much greater flux was obtained with the sodium Diclofenac solution containing the KCl additive compared to the solution without KCl. The delivery rate of Diclofenac can thus be controlled by selecting an appropriate additive to reduce the electric potential difference occurring within the skin.

Diclofenac and 0.1% KCl can be used to manufacture a transdermal patch by employing a sol similar to that used for Procaterol. Table 8 shows a comparison to three Diclofenac products currently on the market. Our patch shows higher delivery.

Thus, a specific embodiment provides a transdermal delivery device including in an active agent layer, Diclofenac and 0.1% KCl, and a sol similar to that used for Procaterol. Table 8 shows a comparison to three Diclofenac products currently on the market. The patch (F26) containing ionizable additive KCl shows higher delivery.

TABLE 8

| | | | | | Taiwan | | | |
|---|---|---|---|---|---|---|---|---|
| | | | | | Panadol | | | |
| | Japan | | Korean | | Topical Oil | | | |
| | V-tape | | R-tape | | Plaster | | patch F26 | |
| | Loading Amount | | | | | | | |
| | (μgcm⁻²) | | | | | | | |
| | 214 | | 2449 | | 429 | | 260 | |
| hr. | Ave. | S.D. | Ave. | S.D. | Ave. | S.D. | Ave. | S.D. |
| 1 | 0.0 | 0.0 | 0.0 | 0.0 | 0.0 | 0.0 | 0.0 | 0.0 |
| 2 | 2.8 | 1.8 | 0.0 | 0.0 | 1.7 | 0.3 | 1.4 | 0.4 |
| 3 | 9.1 | 2.3 | 0.0 | 0.0 | 3.5 | 0.7 | 2.9 | 1.3 |
| 4 | 14.1 | 2.9 | 2.0 | 0.8 | 5.0 | 0.9 | 4.5 | 0.9 |
| 5 | 21.0 | 4.5 | 3.7 | 1.2 | 6.7 | 1.4 | 7.7 | 0.5 |
| 6 | 24.9 | 5.1 | 4.7 | 1.4 | 8.5 | 1.6 | 10.1 | 0.7 |
| 8 | 36.8 | 6.1 | 7.5 | 2.0 | 12.0 | 2.2 | 14.1 | 1.0 |
| 24 | 107.2 | 10.2 | 79.1 | 14.2 | 49.5 | 6.0 | 154.8 | 18.3 |
| μgcm⁻²/h | 4.5 | — | 3.3 | — | 2.1 | — | 6.4 | — |
| Permeation | 50.1 | — | 3.2 | — | 11.5 | — | 59.5 | — |
| Percentage % | | | | | | | | |

3. Ascorbic Acid and Derivatives Thereof

Ascorbic acid is a two-glucoside conductor with high water solubility. Hydrophobic ascorbic acid derivatives have been developed in order to increase the skin permeation of ascorbic acid. However, hydrophobic ascorbic acid derivatives may be combined with a hydrophobic base in which a variety of additives may be used. This may lead to skin irritation, and patches using such formulations may not be well accepted by the public. It is thus described herein a topical formulation (e.g., a hydrophilic lotion) having superior usability, without irritation, without the use of additives by using ascorbic acid 2-glucoside.

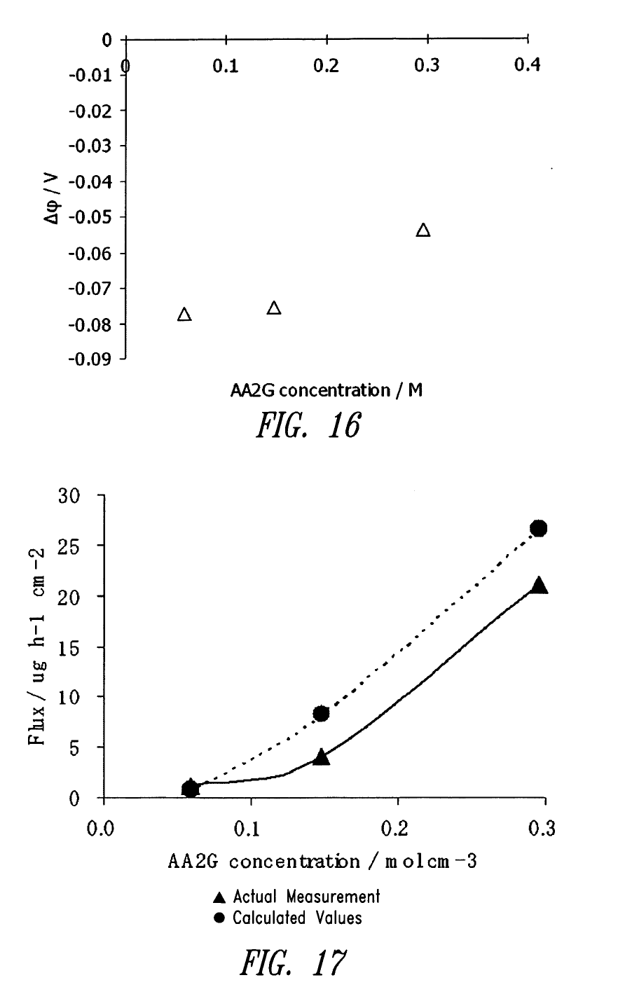

Ascorbic acid 2-glucoside (AA2G) dissociates into AA2G− and H+ ions. FIG. 15 shows a relationship between the concentration of AA2G and AA2G−ions within the skin. FIG. 16 shows the electric potential difference occurring within the skin. An electric potential difference that tends to drive anions from outside of the skin toward the inside of the skin occurs at concentrations of 0.06 M, 0.15 M, and 0.3 M. The electric potential gradient weakens as the concentration becomes higher, however, and it thus becomes more difficult to accelerate the diffusion of AA2G− anions using this potential difference. The reason that the potential difference is high at low concentration is thought to be due to the influence of the ionic concentration difference between physiological saline and AA2G within the skin. Further, different concentrations of AA2G used leads to differences in the movement of H+ and AA2G− within the skin. The electric potential difference found experimentally is thought to occur due to the influence of AA2G− and H+.

It is possible to find the mobility of AA2G− (compared to that of chloride ions) from the film potential, and Table 9 shows results for AA2G at 0.3 M. From this table, the ratio between the mobility of AA2G− and chloride ions is 0.83.

TABLE 9
Concentration of AA2G: 300 mM

| | |
|---|---|
| Concentration | Concentration |
| Donor (mol dm⁻³) | Receiver (mol dm⁻³) |

| Cation 1 | Anion 1 | Cation 1 | Anion 1 |
|---|---|---|---|
| H⁺ | AA2G⁻ | Na⁺ | Cl⁻ |
| 0.296 | 0.296 | 0.15 | 0.15 |

| | |
|---|---|
| Mobility of ion | Mobility of ion |

| Cation 1 | Anion 1 | Cation 1 | Anion 1 |
|---|---|---|---|
| 10 | 0.83 | 1 | 1 |

The flux of AA2G− can then be computed using these results. Table 10 shows results when Eq. 11 is used.

TABLE 10

| | | | | | | | Mobility | |
|---|---|---|---|---|---|---|---|---|
| Membrane | Flux (mol) | Mobility | | | Thickness | | of | Flux |
| Potential | J/mols⁻¹ | ratio | | Faraday | of | Concentration/ | Cl⁻ | J/μg h⁻¹ |
| Δφ/V | cm⁻² | w/— | Charge z | Constant F | Skin d | mol cm³ c | wCl | cm⁻² |
| 0.0537 | 2.18692E−11 | 0.83 | −1 | 96500 | 0.01 | 0.000296 | 1.5E−13 | 26.610497 |
| 0.0753 | 6.82986E−12 | 0.4 | −1 | 96500 | 0.01 | 0.000148 | 1.5E−13 | 8.310569 |
| 0.0772 | 6.9499E−13 | 0.1 | −1 | 96500 | 0.01 | 0.000059 | 1.5E−13 | 0.845664 |

Table 11 shows experimental results for flux measurement. A comparison between the computational results and the experimental results is shown in FIG. 17. Both show a similar trend, and flux may thus be predicted without doing any experiments by using Eq. 11.

TABLE 11

| | time (hr) | | | | Flux |
|---|---|---|---|---|---|
| Concentration/M | 0 | 1 | 3 | 5 | μg h⁻¹ cm⁻² |
| 0.296 | 0 | 25.7 | 121.5 | 211.0 | 21.1 |
| 0.148 | 0 | 5.1 | 29.2 | 40.0 | 4.0 |
| 0.059 | 0 | 2.3 | 7.2 | 12.2 | 1.2 |

4. Lidocaine HCl

Due to the low permeation rate of Lidocaine, is necessary to employ a high concentration of Lidocaine HCl in order to achieve an anesthetic effect. High concentrations of Lidocaine HCl, however, are irritating to the skin. It is thus desirable to develop a patch capable of exhibiting a sufficient anesthetizing effect by effectively delivering Lidocaine into the skin. More specifically, concentrations of Lidocaine HCl that are favorable for permeation can be established according the theoretical model described herein.

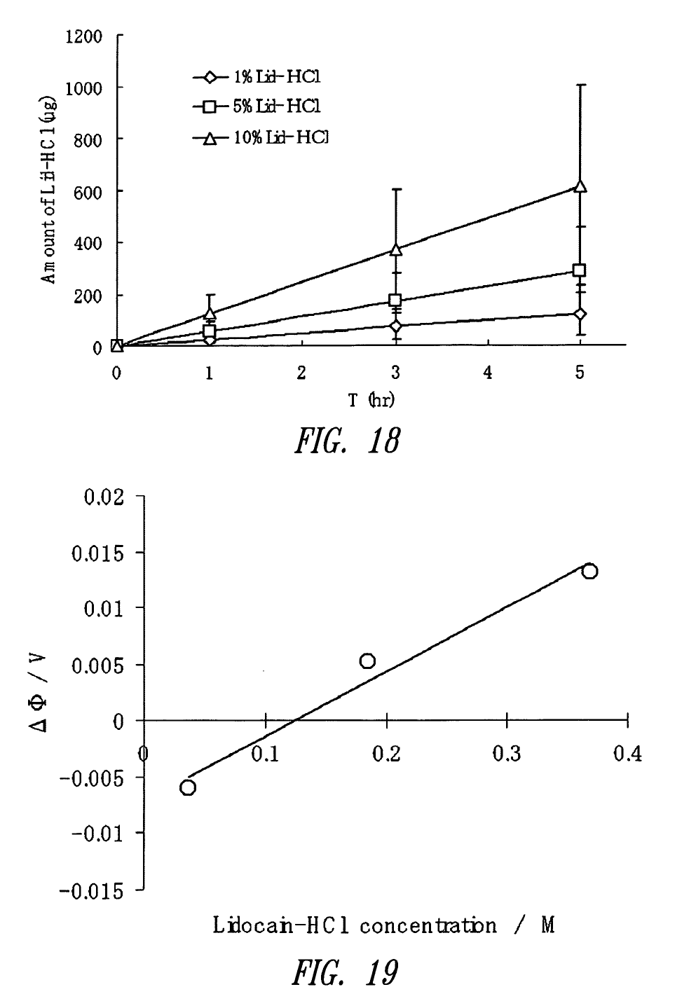

Lidocaine HCl dissociates into Lidocaine cations (protonated Lidocaine) and Cl− ions in water. A relationship between the concentration of Lidocaine HCl and Lidocaine cations delivered within the skin is shown in FIG. 18. FIG. 19 shows the electric potential difference generated within the skin. An electric potential difference that does not tend to drive Lidocaine ions into the skin is found at low concentration (1%), but potential differences that tend to drive Lidocaine ions into the skin occur at higher concentrations (e.g., 5% and 10%).

The mobility of Lidocaine cations with respect to chloride ions can be found from the membrane potential results. Results for 5% Lidocaine (185 mM) are shown in Table 12. Using a value from the table where the membrane potential is the same as actual measurements, the mobility of Lidocaine cations is 0.67 that of chloride ions. Lidocaine cations move relatively slower than chloride ions.

TABLE 12
Concentration of Lid-HCl: 185 mM

| | |
|---|---|
| Concentration | Concentration |
| Donor (mol dm⁻³) | Receiver (mol dm⁻³) |

| Cation 1 | Anion 1 | Cation 1 | Anion 1 |
|---|---|---|---|
| Lid⁺ | Cl⁻ | Na⁺ | Cl⁻ |
| 0.185 | 0.185 | 0.15 | 0.15 |

| | |
|---|---|
| Mobility of ion | Mobility of ion |

| Cation 1 | Anion 1 | Cation 1 | Anion 1 |
|---|---|---|---|
| 0.67 | 1 | 1 | 1 |

| DE | measured membrane potential (V) |
|---|---|
| −0.005237319 | 0.00523047 |

Results of computing Lidocaine cation flux are shown in Table 13, while experimental results are shown in Table 14.

TABLE 13

| | | | | | | | Mobility | |
|---|---|---|---|---|---|---|---|---|
| Membrane | Flux (mol) | Mobility | | | | | of | Flux |
| Potential | J/mols−1 | ratio | | Faraday | Thickness of | Concentration/ | Cl⁻ | J/μg h⁻¹ |
| Δφ/V | cm⁻² | w/— | Charge z | Constant F | Skin d | mol cm³ c | wCl | cm⁻² |
| 0.005334558 | 2.69195E−12 | 2.15 | 1 | 96500 | 0.01 | 0.000296 | 1.5E−13 | 2.624328 |
| −0.005237319 | 5.14038E−12 | 0.67 | 1 | 96500 | 0.01 | 0.000148 | 1.5E−13 | 5.011253 |
| −0.012928312 | 7.93717E−12 | 0.45 | 1 | 96500 | 0.01 | 0.000059 | 1.5E−13 | 7.737789 |

TABLE 14

| | time (hr) | | | | Flux |
|---|---|---|---|---|---|
| Concentration/M | 0 | 1 | 3 | 5 | μg h⁻¹ cm⁻² |
| 0.296 | 0 | 23.9 | 71.8 | 119.6 | 0.7 |
| 0.148 | 0 | 57.4 | 172.2 | 287.0 | 1.8 |
| 0.059 | 0 | 122.5 | 367.5 | 612.4 | 3.8 |

Calculations were made assuming a skin thickness of 0.01 cm and a chloride ion mobility of 1.5×10⁻¹³. FIG. 18 shows the measurements of the actual amount of Lidocaine aqueous solution delivered to hairless mouse skin over time at a variety of concentrations.

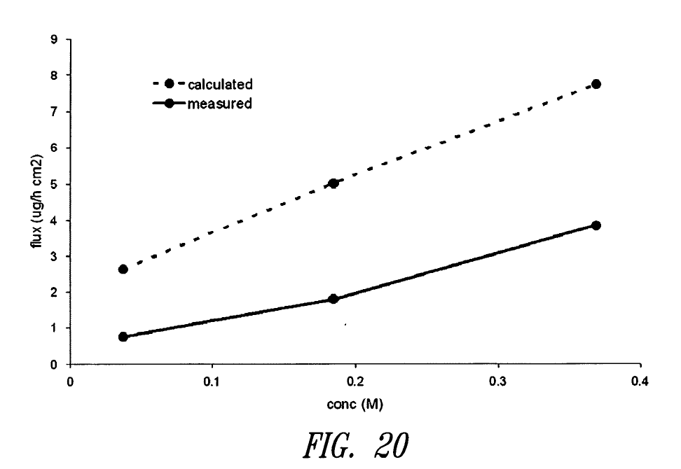

FIG. 20 shows a comparison of computed and actual experimental values. Both show a similar trend, indicating that Eq. 11 can be used to predict the amount of flux independently of performing experiments.

### Topical Formulations

In certain embodiments, the active agent layer described in connection with the transdermal delivery device can be hydrated to form topical formulations. The topically formulation can be applied directly and freely to the skin of a subject. Thus, certain embodiments provide a topical formulation including a thickening agent and an ionized active agent, as described herein, in combination with an aqueous medium, wherein the topical formulation is substantially oil-free. The topical formulations are typically formulated into spreadable forms (e.g., plasters and paste) according to known methods in the art. Various additives, including permeation enhancers, antioxidants can be further combined with the topical formulation.

In certain embodiments, the ionized active agent can be based on any of the ionizable active agents described herein. One specific embodiment provides a topical formulation comprising Procaterol cation (e.g., Procaterol HCl). For example, the topical formulation includes HPC, Procaterol, urea, and water to provide an aqueous-based formulation. Another specific embodiment provides a topical formulation comprising Lidocaine cations (e.g., Lidocaine HCl). A further specific embodiment provides a topical formulation comprising AA2G anion. A further specific embodiment provides a topical formulation comprising Diclofenac anion (e.g., sodium Diclofenac). As in the passive patch application, ionized additives can be added to adjust the electrical potential difference. Advantageously, the absence of oil in the topical formulation promotes long-term stability of the ionized active agent in the topical formulation.

The topical formulation can be formulated and used according to known methods in the art.

### Methods of Use and Making

The transdermal delivery device and topical formulations described herein can be constructed by known methods in the art.

Typically, an active agent layer can be prepared by dispersing an ionizable active agent in a viscous sol based on a thickening agent (e.g., HPC). This was applied on top of a backing substrate, e.g., polyethylene terephthalate (PET) film. The backing substrate can be in the shape of a patch, tape, disc, and so forth.

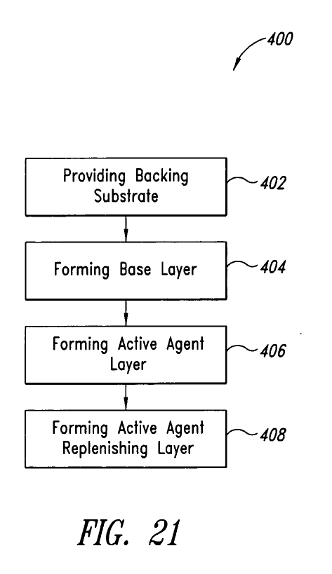

FIG. 21 shows an exemplary method 400 for manufacturing the delivery devices 10a, 10b, and 10c, which hereinafter are collectively referred to as delivery device 10. Various components, features, layers, etc. of the delivery device 10 are referred to herein below by reference numerals, which generally correspond to various components, features, layers of delivery devices 10a, 10b, and 10c having the same reference numeral and a letter appended thereto.

At 402, a backing substrate 12 is provided. The backing substrate 12 has a first surface 13 and an opposed second surface 125.

At 404, a base layer 14 having a thermoplastic resin is formed on the first surface 13 of the backing substrate 12. In some embodiments, the base layer 16 includes a poly(ethylene terephthalate) material

At 406, an active agent layer 16 is formed on the base layer 14 on the first surface 13 of the backing substrate 12. The active agent layer 16 may include a thickening agent, a humectant, and a therapeutically effective amount of a β2-adrenoreceptor agonist (or β₂-adrenoreceptor stimulant) or derivative or pharmaceutically acceptable salt thereof.

In some embodiments, forming an active agent layer 16 on the base layer 14 on the first surface of the backing substrate 12 includes spin-coating a composition thereon. Compositions that may be spin-coated include, but are not limited to: a composition having a thickening agent, a humectant, and a therapeutically effective amount of an ionizable active agent. For example, the active agent layer may comprise hydroxypropyl cellulose, glycerol or urea, and Procaterol HCl or other β₂-adrenoreceptor agonist in various amounts such as an amount ranging from about 0.1 wt % to about 5 wt % of the total composition.

At 408, which in some embodiments is optional, an active agent replenishing layer 18 adjacent to the active agent layer 16 is formed. The active agent replenishing layer 18 may be spin coated onto the active agent layer and may include an ion exchange material and a sufficient amount of the ionizable active agent (e.g., β₂-adrenoreceptor agonist) to maintain a weight percent composition of about 0.1 wt % to about 5 wt % in the active agent layer 16.

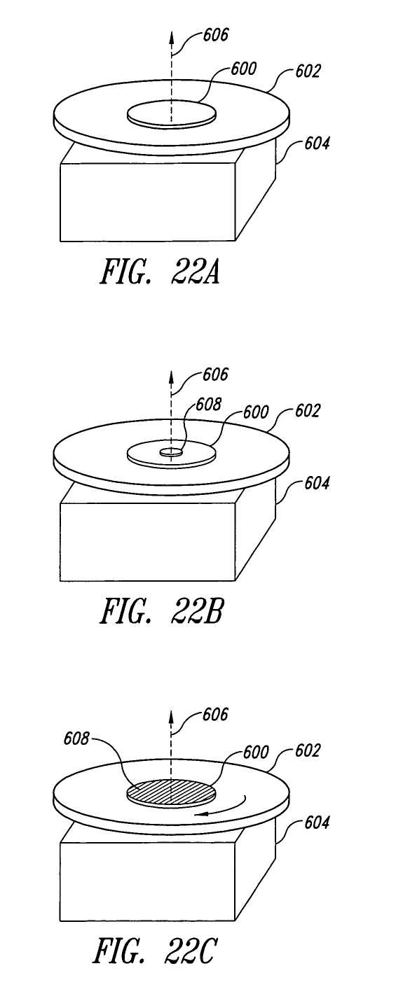

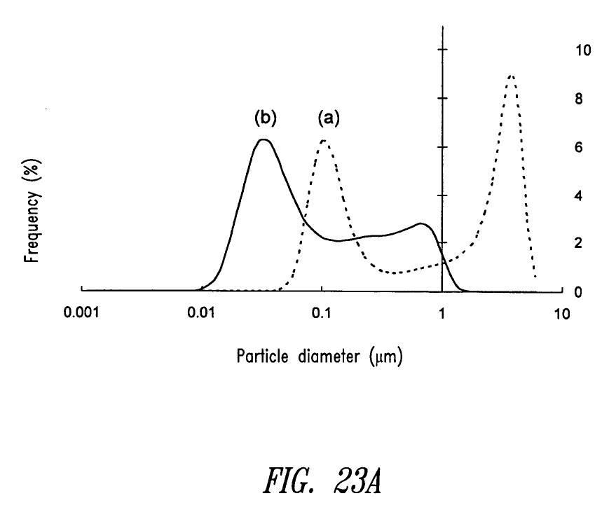

FIGS. 22A-22C show a spin-coating process of a layer of material 600 according to one illustrated embodiment. In FIG. 22A, the layer of material is disposed on a spinable disc 602 that is controllably driven by rotation device 604. The rotation device 604 may rotate the disc 602 (and the layer of material 600 placed thereon) about an axis 606. In some embodiments, the rotation device 602 is controllable/variable such that the rate at which the disc 600 rotates is controllable.

In FIG. 22B, an amount of an active agent 608 is disposed, proximal to the axis 606, on to the layer of material 600. In some embodiments, the active agent 608 may be disposed on the layer of material 600 while the disc 602 is rotating. In other embodiments, the active agent 608 may disposed on the disc 602 while the disc 602 is not rotating, and then rotation device 604 may be actuated to cause the disc to rotate.

In FIG. 22C, the active agent 608 is shown spread out over the layer of material 600 in response to the rotation of the disc 602. Spin-coating the active agent 608 onto the layer of material 600 provides an even coating of the active agent 608 onto the layer of material 600. In some embodiments, the layer of material 600 may be the base layer 14 without the backing substrate 12, i.e., prior to the base layer 14 being applied to the backing substrate 12. In other embodiments, the layer of material 600 may be the base layer 14 and the backing substrate 12.

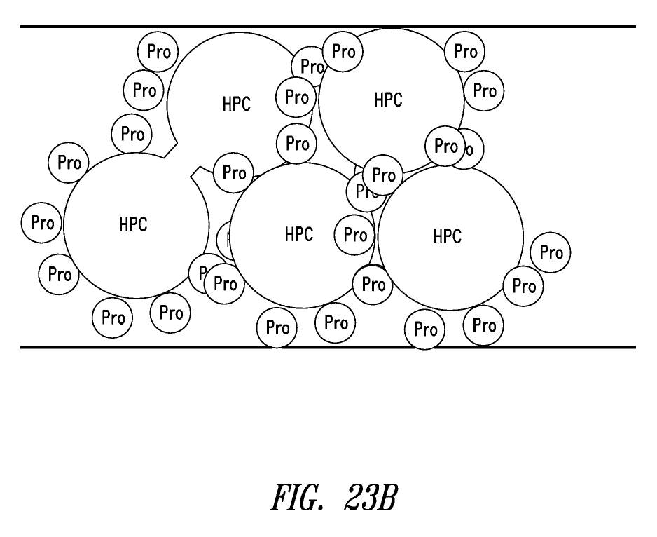

Sol structures can be investigated by dynamic light scattering (DLS). Scattered laser light can be used to identify the state of the HPC contained in the sol. FIG. 23A shows a DLS measurement spectra plots. It can be seen that different spectra are obtained for solutions containing only HPC (b) versus solutions containing HPC, Procaterol, and glycerol (a). HPC interacts with Procaterol and/or glycerol, forming aggregates. Although it's important for the sol to contain aggregates in order to maintain a certain level of viscosity, aggregates become an impediment to ionic separation of Procaterol and/or release of Procaterol from the patch. FIG. 23B shows a cross sectional view of an active agent layer illustrating the interactions of HPC and Procaterol HCl according to one illustrated embodiment.

The aggregate state between HPC and Procaterol becomes an important factor in regulating the active agent sol in the patch. Procaterol HCl is cationic, and HPC is highly hydrophilic. HPC may also be considered to have anionic properties when its pH is acidic, thus leading to the development of aggregates.

For a topical formulation, the ionizable active agent (e.g., AA2G) can be formulated into lotions, cream, emulsions according to known methods in the art.

The ionizable active agent described herein can thus be delivered transdermally in a therapeutically effective amount for treatment of various conditions. Certain embodiments describe method of treating a condition associated with an obstructive respiratory ailment by applying a transdermal delivery device to the skin of a subject, the transdermal delivery device including an active agent layer comprising a β-adrenoreceptor stimulant such as Procaterol HCl.

Obstructive respiratory ailments including, for example, asthma (e.g., allergic asthma, bronchial asthma, and intrinsic asthma), bronchoconstrictive disorders, chronic obstructive pulmonary disease, and the like, affect millions of children and adults worldwide. These ailments are typically characterized by bronchial hyper-responsiveness, inflammation (e.g., airway inflammation), increased mucus production, and/or intermittent airway obstruction, often in response to one or more triggers or stresses. For example, obstructive respiratory ailments may result from exposure to an environmental stimulant or allergen, air pollutants, cold air, exercise or exertion, emotional stress, and the like. In children, the most common triggers are viral illnesses such as those that cause the common cold. Signs of an asthmatic episode include wheezing, shortness of breath, chest tightness, coughing, rapid breathing (tachypnea), prolonged expiration, a rapid heart rate (tachycardia), rhonchous lung sounds, over-inflation of the chest, and the like.

Ionizable active agents belong to the class of amine-containing β-adrenoreceptor stimulants can be formulated into an active agent layer and delivered transdermally into a subject according to various embodiments. β₂-receptors are generally located on a number of tissues including blood vessels, bronchi, gastro intestinal tract, skeletal muscle, liver, and mast cell. Typically β₂-adrenoreceptor agonist act on the β₂-adrenergic receptor eliciting smooth muscle relaxation resulting in dilation of bronchial passages (bronchodilation), relaxation of the gastro intestinal tract, vasodilation in muscle and liver, relaxation of uterine muscle and release of insulin, glycogenolysis in the liver, tremor in skeletal muscle, inhibition of histamine release from mast cells, and the like. β₂-adrenoreceptor agonists are useful for treating asthma and other related bronchospastic conditions, and the like. β-receptor antagonists are also useful as anti-hypertensive agents.

Thus, one embodiment provides a method for treating a condition associated with an obstructive respiratory ailment in a subject comprising: applying to the subject's skin a passive transdermal delivery device comprising: a backing substrate; and an active agent layer, wherein the active agent layer is substantially anhydrous and oil-free and includes a thickening agent and an ionizable active agent, and wherein the ionizable active agent is electrically neutral in the active agent layer and dissociates into an ionized active agent upon contacting an aqueous medium; and allowing the ionizable active agent to dissociate into the ionized active agent.

In certain embodiments, the method comprises contacting the ionizable active agent to sweat of the subject's skin to produce the ionized active agent.

In other embodiments, the ionizable active agent is a β-receptor antagonist. In a specific embodiment, the ionizable active agent is Procaterol HCl.

In some embodiments, at least 50% of the Procaterol HCl is delivered through the skin of the subject within a 24 hour period.

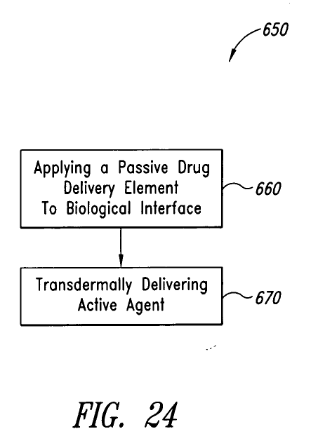

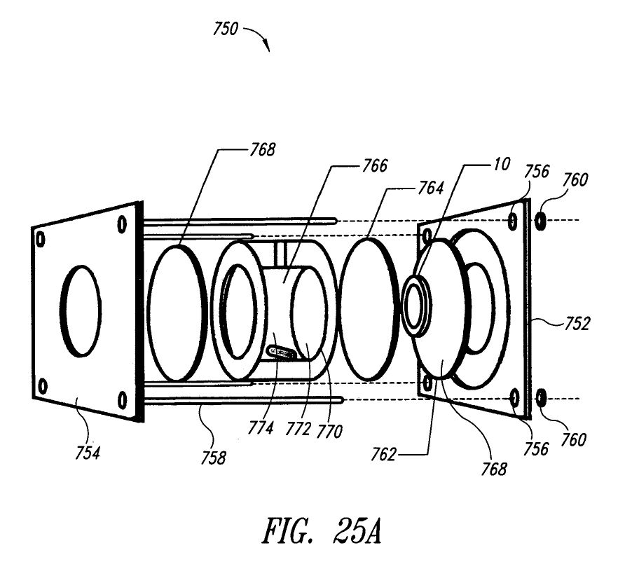

FIG. 24 shows an exemplary method 650 of treating a condition associated with an obstructive respiratory ailment.

At 660, a transdermal delivery device comprising from about 25 μg to about 100 μg of an active agent having β-adrenoreceptor stimulant activity is applied to a biological interface of a subject. A skill artisan can select an appropriate amount of an active agent, however, based on the condition to be treated or the pharmacokinetics, or other criteria or properties of the active agent to achieve the desired effect (e.g., an amount sufficient to alleviate the condition associated with an obstructive respiratory ailment).

At 670, the active agent having β-adrenoreceptor stimulant activity is delivered to the biological interface in an amount sufficient to alleviate the condition associated with an obstructive respiratory ailment.

In some embodiments, transdermally delivering the active agent having β-adrenoreceptor stimulant activity to the biological interface includes transferring a therapeutically effective amount of a β₂-adrenoreceptor agonist to the biological interface of the subjected via diffusion. In some embodiments, transdermally delivering the active agent having β-adrenoreceptor stimulant activity to the biological interface includes transferring a therapeutically effective amount of a β₂-adrenoreceptor agonist selected from Procaterol HCl, Procaterol HCl hemihydrate, or a derivative or pharmaceutically acceptable salt thereof to the biological interface of the subjected.

In the description above, active agents such as ionic exchange materials were described as being disposed on a patch for being applied to the skin of a subject. In alternative embodiments, active agents including, but not limited to, ion exchange materials may be in the form of a powder or cream that may be applied to the skin of a subject.

The various embodiments described herein are further illustrated by the following non-limiting examples.

### EXAMPLES

1. In-Vitro Permeation Testing

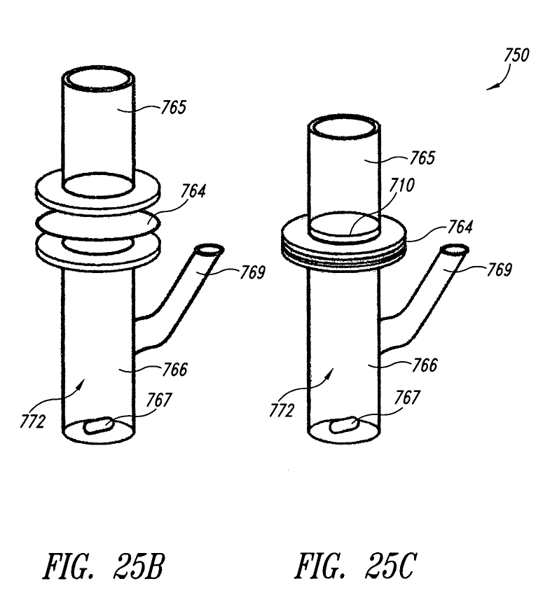

Delivery devices 10a, 10b, and 10c, which are hereinafter collectively referred to as delivery device 10, may be tested using both in vitro and in vivo. In vitro testing may be performed using a passive diffusion-testing device such as a Kelder cell or a Franz cell, among other types of testing devices. FIGS. 25A, 25B, and 25C show multiple exemplary passive diffusion measuring devices 750 used for testing a delivery device 10.

The passive diffusion measuring device 750 includes a first end plate 752 and a second end plate 754. A plurality of coupling features such as holes 756 are formed on the first end plate 752. The second end plate 754 includes a number of coupling features such as arms 758, which are complementarily aligned with the holes 756. The holes 756 are sized and shaped to receive at least a portion of the arms 758. In operable position, a portion of the arms 758 extend through the holes 756, and the arms 758 receive fasteners 760, which hold the arms in place.

Sandwiched between the first end plate 752 and the second end plate 754 is a first cap 762, the delivery device 10, a permeable membrane 764, a reservoir 766, and a second cap 768. The first cap 762 abuts the first end plate 752, and the second cap 768 abuts the second end plate 754. The first cap 762 and the second cap 768 may be non-permeable and made from a material such as silicon rubber.

The delivery device 10 interposes first cap 762 and the permeable membrane 764. In the experiments described below, the permeable membrane 764 is a piece of human skin or animal skin (e.g., hairless mouse skin obtained from “HOS hr-1” male mice).

Interposing the permeable membrane 764 and the second cap 768 is the reservoir 766. The reservoir 766 is made from a non-permeable material such as rubber, silicon rubber, glass, and the like. The reservoir 766 may be generally cylindrical with an open end 770 that is in fluidic communication with a generally hollow interior 772. The open end 770 abuts the permeable membrane 764. A fluid 774 such as Phosphate Buffered Saline (PBS) is disposed in the hollow interior 772. At the open end 770, the fluid 774 contacts the permeable membrane 764. The active agent in the delivery device diffuses through the permeable membrane 764 in to the fluid 774. In the experiments described below, the reservoir 766 may hold about 4 milliliters of the fluid 774.

2. In-Vitro Testing Conditions and Measurements

Typically, 17 ml of phosphate buffered saline (PBS, sold by Wako Pure Chemical Industries) was injected into the receptor cell, and a 10 mm stirring bar was used to agitate the solution during the test. The Franz cell was placed in an incubator (made by ESPEC, model LH-113) with the temperature set to 32° C. and the humidity set to 70%. Samples were typically extracted from the cell at predetermined times using a 200 μl Gilson Pipetman. 200 μl of PBS was then added to the cell after each sampling operation.

For measuring the active agent (e.g., Procaterol cation) permeated, a standard solution with known concentration can be prepared and compared with the concentration measured. Using Procaterol HCl as an example, 50 mg of Procaterol HCl (97.25% anhydrous) was accurately measured out, and then added to water to form 50 ml of solution (“Procaterol concentrate liquid”). The standard concentrate was then diluted (“Procaterol standard solution”) and used as a mobile phase for high performance (or pressure) liquid chromatography (HPLC). The Procaterol concentrate liquid was sealed in a light shielding bottle and stored in a refrigerator. 10 μl of each test sample and 10 μl of the standard solution was measured using HPLC, and Procaterol peak areas Aₜ (test samples) and Aₛ (standard solution) were determined for each sample. Procaterol HCl masses were then found for each test sample using the following equation:

Amount of Procaterol HCl in test solution (g/μl)=amount of anhydrous Procaterol in standard concentrate liquid×Aₜ/Aₛ×1.0276, where 1.0276 is the ratio between the molecular weight of ½ hydrated Procaterol HCl/the molecular weight of anhydrous Procaterol HCl=335.83/326.82

Below are an exemplary condition and instrument for measuring the concentration of Procaterol cations permeated:

Model: Shimazu HPLC LC-2010A HT

Column: Shinwa Chemical Industries, Ltd.

model STRUCTURE ODS-II

150 mm length×4.6 mm internal diameter

Temperature: 40° C.

Mobile phase: 5 m-mol dm⁻³ of a mixture of pentane sulfonic acid/methanol/acetic acid (76:23:1)

Flow rate: 1 ml min⁻¹

Amount injected: 10 μl

Unless indicated otherwise, hairless mouse skin obtained from “HOS: hr-1”, 5 weeks old male mice:

Set up glass chambers to run at 32.5° C.

Approximately 3.4 ml of DPBS in chamber

Chambers 1, 2, 3, 4, 5 TT spincoat

Chambers 6, 7 PP-HPC

Chambers 8, 9 PET-HPC

3. Exemplary Patch Preparations

Preparation 1: 23.5 μg Procaterol patch (1.13 cm²) was made by spin-coating an active agent layer 16 composition comprising 2.5 wt % Procaterol-Hydrochloride (HCl), 0.5 wt % HPC in a 10 wt % Glycerol solution on to a 12 mm diameter PET base layer 16 on a backing sheet (3M).

Preparation 2: 2.5 mg Procaterol patches were made by adding 100 μL of a 25 mg/ml Procaterol/10 wt % glycerol solution/ to a 10 mm diameter single PET-Klucel layer disc.

Preparation 3: 0.75 mg Procaterol patches were made by adding 30 μL of a 25 mg/ml Procaterol/10 wt % glycerol solution/ to a 12 mm diameter two PP-Klucel layer disc.

### Example 1

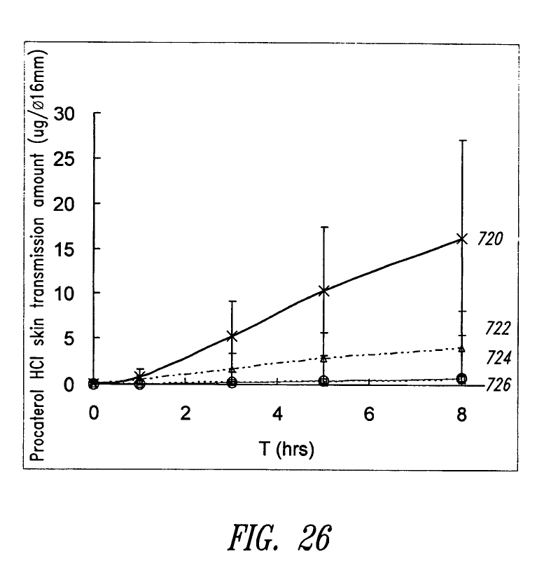

In Example 1, before testing the delivery device 10, sixteen tests were performed at four different agent concentrations (four tests (#1, #2, #3, and #4) for each concentration of active agent) using Procaterol HCl in order to investigate the transport of Procaterol cation into and through skin along a concentration gradient. A Franz cell was used at 32° C. using hairless mouse skin as a permeable membrane. 720 corresponds to the average delivery of a 5 wt % Procaterol-HCl concentration, 722 corresponds to the average delivery of a 2.5 wt % Procaterol-HCl concentration, 724 corresponds to the average delivery of a 1 wt % Procaterol-HCl concentration, and 726 corresponds to the average delivery of a 0.5 wt % Procaterol-HCl concentration. FIG. 26 shows the average amount of active agent delivered to the reservoir 772, which has PBS fluid 74 therein, versus time for the four agent concentrations 720, 722, 724, and 726. It can be seen that the amount of Procaterol delivered through the skin increases over time. Further, it can also be seen that the amount of Procaterol delivered increases with increased Procaterol concentration. To deliver a medically effective amount of Procaterol through the skin, the concentration of the Procaterol solution must be equal to or greater than a certain threshold concentration. A sufficient amount of Procaterol dissolved in water was used in this experiment, thus leading to a rather large Procaterol delivery speed. It is therefore possible to deliver Procaterol through the skin, provided that the solution exists in proximity to the surface of the skin. Table 16 shows the details of the test delivery devices 720-726.

TABLE 15
Total transmission amount

| Test ID (concentration) | Sample ID | 0 hrs. | 1 hr. | 3 hrs. | 5 hrs. | 8 hrs. |
|---|---|---|---|---|---|---|
| 720 (5.0%) | #1 | 0.0 | 0.0 | 2.7 | 6.1 | 10.1 |
| | #2 | 0.0 | 1.3 | 8.9 | 17.4 | 27.9 |
| | #3 | 0.0 | 0.0 | 1.1 | 2.6 | 4.4 |
| | #4 | 0.0 | 1.6 | 8.3 | 15.4 | 22.7 |
| | Ave. | 0.0 | 0.7 | 5.3 | 10.4 | 16.3 |
| | SD | 0.0 | 0.9 | 3.9 | 7.2 | 10.9 |
| 722 (2.5%) | #1 | 0.0 | 0.8 | 3.2 | 5.5 | 7.8 |
| | #2 | 0.0 | 0.8 | 3.2 | 5.0 | 7.4 |
| | #3 | 0.0 | 0.0 | 0.0 | 0.0 | 0.2 |
| | #4 | 0.0 | 0.0 | 0.4 | 0.7 | 0.9 |
| | Ave. | 0.0 | 0.4 | 1.7 | 2.8 | 4.1 |
| | SD | 0.0 | 0.5 | 1.7 | 2.9 | 4.1 |
| 724 (1.0%) | #1 | 0.0 | 0.0 | 0.3 | 0.6 | 1.2 |
| | #2 | 0.0 | 0.0 | 0.0 | 0.1 | 0.2 |
| | #3 | 0.0 | 0.0 | 0.1 | 0.2 | 0.5 |
| | #4 | 0.0 | 0.0 | 0.3 | 0.5 | 0.9 |
| | Ave. | 0.0 | 0.0 | 0.2 | 0.4 | 0.7 |
| | SD | 0.0 | 0.0 | 0.1 | 0.3 | 0.5 |
| 726 (0.5%) | #1 | 0.0 | 0.0 | 0.3 | 0.8 | 1.3 |
| | #2 | 0.0 | 0.0 | 0.1 | 0.4 | 0.8 |
| | #3 | 0.0 | 0.0 | 0.1 | 0.3 | 0.5 |
| | #4 | 0.0 | 0.0 | 0.2 | 0.4 | 0.6 |
| | Ave. | 0.0 | 0.0 | 0.2 | 0.5 | 0.8 |
| | SD | 0.0 | 0.0 | 0.1 | 0.2 | 0.3 |

It is generally possible to manufacture a transdermal delivery patch using a hydrophilic gel polymer matrix such as polyvinyl pyrrolidone or polyvinyl alcohol. Procaterol is a hydrophilic active agent, however, and thus smooth release from within a polymer matrix may not always be possible.

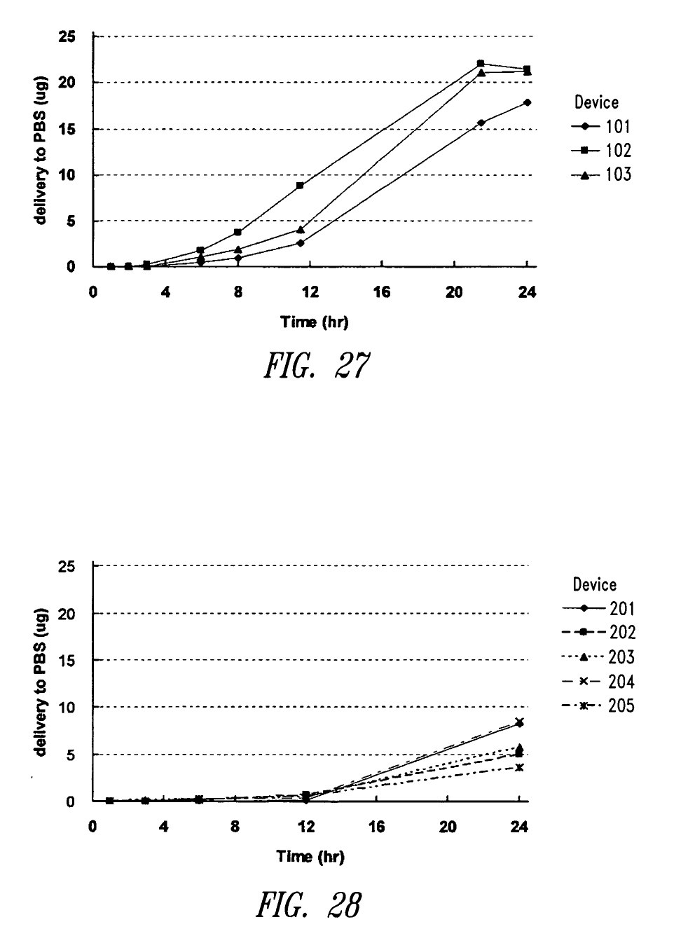

FIGS. 27-32 show in vitro test results for various embodiments of the delivery device 10 under various test conditions and for various concentrations of agents.

Examples 2-7 described below generally employed a high viscosity sol solution in order to hold Procaterol. Several wt % of hydroxypropyl cellulose (HPC) was dissolved in water in order to form an active agent containing sol. Procaterol HCl was then dissolved in the sol. The sol was applied to a PET sheet, forming a patch. Glycerol (generally 10 wt %) was added to, among other things, promote delivery. The amount of active agent solution applied to the PET contained approximately 20 μg/cm² of Procaterol. In some tests, a composition of HPC and glycerol was made and allowed to repose for a given period of time, such as a day or two. In some situations, the period of repose may be shorter or longer.

Patches were applied to the skin (frozen or raw) of a hairless mouse, and the amount of Procaterol delivered was measured using the previously described Frantz cell setup, with the patch replacing the solution. Experiments 2-7 show that the amount of Procaterol on the donor side increases over time, and passes through the skin. Although Examples 2-7 can measure the amount of Procaterol delivered through the skin, the actual delivery mechanism of Procaterol may be complex.

### Example 2

One lot of six delivery devices was prepared according to the embodiment shown in FIG. 4A-4B. The surface area for each respective active agent layer 16 was approximately 1.12 cm². In Example 2, three of the delivery devices were tested in the passive diffusion measuring device 750 (FIG. 25A), and frozen skin was used for the permeable membrane 764. Each respective active agent layer 16 included HPC (approximately 1 wt %) and Procaterol-HCl (approximately 1 wt %); each respective replenishing layer 18 included HPC (approximately 1 wt %). FIG. 27 shows the amount of active agent delivered to the reservoir 772, which has PBS fluid 774 therein, versus time for three delivery devices, individually referenced as test devices 101, 102, and 103. Table 16A shows flux rate measured for the test devices 101, 102, and 103, calculated using data taken at 11.5 hours. Three further test devices from the one lot, individually referenced as test devices 104, 105, and 106, were analyzed to determine the amount of active agent present in each device. Table 16B shows active agent amount and concentration details for the delivery devices 104, 105, and 106.

TABLE 16A

| | Flux rate at 11.5 hr |
|---|---|
| | (μg/hr/cm²) |
| Delivery Device | 0.23 |
| 101 | |
| Delivery Device | 0.82 |
| 102 | |
| Delivery Device | 0.38 |
| 103 | |
| Ave. | 0.48 |
| S.D. | 0.31 |

TABLE 16B

| | Amount Of Active | Density of Active |
|---|---|---|
| | Agent (μg) | Agent (μg/cm²) |
| Test Device 104 | 21.05 | 18.63 |
| Test Device 105 | 23.88 | 21.12 |
| Test Device 106 | 23.33 | 20.65 |
| Ave. | 22.75 | 20.14 |
| S.D. | 1.50 | 1.33 |

### Example 3

In Example 3, one lot of eight delivery devices was prepared according to the embodiment shown in FIGS. 1-2B. The surface area for each respective active agent layer 16 was approximately 1.12 cm². In Example 3, the delivery devices were tested in the passive diffusion measuring device 750 (FIG. 25A), and raw skin was used for the permeable membrane 764. Each respective active agent layer 16 included HPC (approximately 1 wt %) and Procaterol-HCl (approximately 1 wt %). FIG. 28 shows the amount of active agent delivered to the reservoir 772, which has PBS fluid 774 therein, versus time for five delivery devices, individually referenced as test delivery devices 201, 202, 203, 204, and 205. Table 17A shows flux rate measured for the test devices 201, 202, 203, 204, and 205, calculated using data taken at 12.0 hours. Three further test devices from the one lot, individually referenced as test devices 206, 207, and 208, were analyzed to determine the amount of active agent present in each device. Table 17B shows active agent amount and concentration details for the delivery devices 206, 207, and 208.

TABLE 17A

| | Flux rate at |
|---|---|
| | 12.0 hr |
| | (μg/hr/cm²) |
| Delivery Device 201 | 0.01 |
| Delivery Device 202 | 0.05 |
| Delivery Device 203 | 0.04 |
| Delivery Device 204 | 0.02 |
| Delivery Device 205 | 0.04 |
| Ave. | 0.03 |
| S.D. | 0.02 |

TABLE 17B

| | Amount Of Active Agent | Density of Active Agent |
|---|---|---|
| | (μg) | (μg/cm²) |
| Delivery Device 206 | 11.14 | 9.87 |
| Delivery Device 207 | 10.96 | 9.7 |
| Delivery Device 208 | 10.40 | 9.2 |
| Ave. | 10.84 | 9.59 |
| S.D. | 0.39 | 0.35 |

### Example 4

In Example 4, one lot of ten delivery devices was prepared according to the embodiment shown in FIGS. 1-2B. The surface area for each respective active agent layer 16 was approximately 1.12 cm². In Example 4, the delivery devices were tested in the passive diffusion measuring device 750 (FIG. 25A), and raw skin was used for the permeable membrane 764. Each respective active agent layer 16 included glycerol (approximately 10 wt %), HPC (approximately 0.5 wt %) and Procaterol-HCl (approximately 2.5 wt %). FIG. 29 shows the amount of active agent delivered to the reservoir 772, which has PBS fluid 774 therein, versus time for five delivery devices, individually referenced as devices 301, 302, 303, 304, and 305. Table 18A shows flux rate measured for the test devices 301-305, calculated using data taken at 12.0 hours. Five further test devices from the one lot, individually referenced as test devices 306-310, were analyzed to determine the amount of active agent present in each device. Table 18B shows active agent amount and concentration details for the delivery devices 306-310.

TABLE 18A

| | Flux rate at |
|---|---|
| | 12.0 hr |
| | (μg/hr/cm²) |
| Delivery Device 301 | 0.19 |
| Delivery Device 302 | 0.08 |
| Delivery Device 303 | 0.54 |
| Delivery Device 304 | 0.54 |
| Delivery Device 305 | 0.08 |
| Ave. | 0.29 |
| S.D. | 0.24 |

TABLE 18B

| | Amount Of Active Agent | Density of Active Agent |
|---|---|---|
| | (μg) | (μg/cm²) |
| Delivery Device 306 | 35.29 | 31.23 |
| Delivery Device 307 | 26.09 | 23.09 |
| Delivery Device 308 | 19.98 | 17.68 |
| Delivery Device 309 | 18.57 | 16.43 |
| Delivery Device 310 | 35.46 | 31.38 |
| Ave. | 27.08 | 23.96 |
| S.D. | 8.08 | 7.15 |

### Example 5

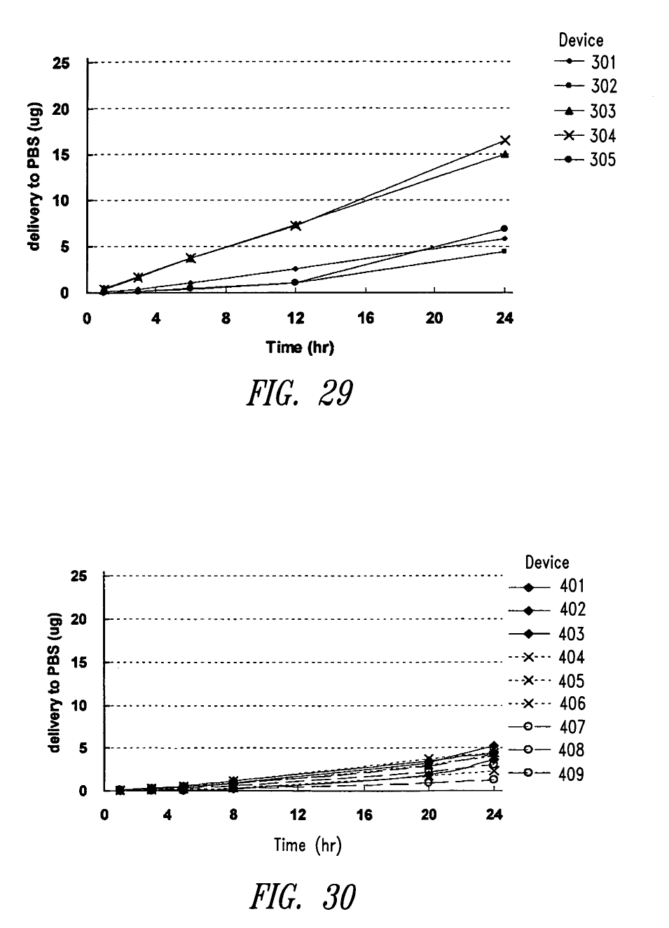

In Example 5, eighteen delivery devices were prepared according to the embodiment shown in FIGS. 1-2B. The surface area for each respective active agent layer 16 was approximately 1.12 cm². In Example 5, the delivery devices were tested in the passive diffusion measuring device 750 (FIG. 25A), and frozen skin was used for the permeable membrane 764. Each respective active agent layer 16 included glycerol (approximately 10 wt %), HPC (approximately 0.5 wt %) Procaterol-HCl (approximately 2.5 wt %), and a buffer solution. Three different pH value buffer solutions were used. FIG. 30 shows the amount of active agent delivered to the reservoir 772, which has PBS fluid 774 therein, versus time for nine delivery devices, individually referenced as devices 401-409. Table 19A shows flux rate measured for the test devices 401, 402, and 403, which used a pH 4.0 buffer solution. The flux rates were calculated using data taken at 8.0 hours. Table 19B shows flux rate measured for the test devices 404, 405, and 406, which used a pH 5.0 buffer solution. The flux rates were calculated using data taken at 8.0 hours. Table 19C shows flux rate measured for the test devices 407, 408, and 409, which used a pH 6.0 buffer solution. The flux rates were calculated using data taken at 8.0 hours. Nine further test devices from the one lot, individually referenced as test devices 410-418, were analyzed to determine the amount of active agent present in each device. Table 19D shows the details of the active agent amount and concentration for the delivery devices 410, 411, and 412, which used the pH 4.0 buffer solution. Table 19E shows active agent amount and concentration details for the delivery devices 413, 414, and 415, which used the pH buffer 5.0 solution. Table 19F shows active agent amount and concentration details for the delivery devices 416, 417, and 418, which used the pH buffer 6.0 solution.

TABLE 19A

| | pH of Buffer | Flux rate at 8.0 hr |
|---|---|---|
| | Solution | (μg/hr/cm²) |
| Delivery Device 401 | 4.0 | 0.13 |
| Delivery Device 402 | 4.0 | 0.03 |
| Delivery Device 403 | 4.0 | 0.11 |
| Ave. | | 0.09 |
| S.D. | | 0.05 |

TABLE 19B

| | pH of Buffer | Flux rate at 8.0 hr |
|---|---|---|
| | Solution | (μg/hr/cm²) |
| Delivery Device 404 | 5.0 | 0.04 |
| Delivery Device 405 | 5.0 | 0.10 |
| Delivery Device 406 | 5.0 | 0.13 |
| Ave. | | 0.09 |
| S.D. | | 0.04 |

TABLE 19C

| | pH of Buffer | Flux rate at 8.0 hr |
|---|---|---|
| | Solution | (μg/hr/cm²) |
| Delivery Device 407 | 6.0 | 0.07 |
| Delivery Device 408 | 6.0 | 0.02 |
| Delivery Device 409 | 6.0 | 0.09 |
| Ave. | | 0.06 |
| S.D. | | 0.04 |

TABLE 19D

| | pH of Buffer | Amount Of | Density of Active |
|---|---|---|---|
| | Solution | Active Agent (μg) | Agent (μg/cm²) |
| Delivery Device 410 | 4.0 | 18.69 | 16.54 |
| Delivery Device 411 | 4.0 | 18.52 | 16.39 |
| Delivery Device 411 | 4.0 | 18.52 | 16.39 |
| Ave. | | 18.52 | 16.39 |
| S.D. | | 0.17 | 0.15 |

TABLE 19E

| | pH of Buffer | Amount Of | Density of Active |
|---|---|---|---|
| | Solution | Active Agent (μg) | Agent (μg/cm²) |
| Delivery Device 413 | 5.0 | 20.08 | 17.77 |
| Delivery Device 414 | 5.0 | 20.08 | 17.77 |
| Delivery Device 411 | 5.0 | 18.52 | 16.39 |
| Ave. | | 20.41 | 18.06 |
| S.D. | | 0.57 | 0.51 |

TABLE 19F

| | pH of Buffer | Amount Of | Density of Active |
|---|---|---|---|
| | Solution | Active Agent (μg) | Agent (μg/cm²) |
| Delivery Device 416 | 6.0 | 25.06 | 22.18 |
| Delivery Device 417 | 6.0 | 25.06 | 22.18 |
| Delivery Device 411 | 6.0 | 18.52 | 16.39 |
| Ave. | | 24.72 | 21.88 |
| S.D. | | 0.59 | 0.52 |

### Example 6

In Example 6, fourteen delivery devices were prepared according to the embodiment shown in FIGS. 1-2B. The surface area for each respective active agent layer 16 was approximately 1.12 cm². In experiment 6, the delivery devices were tested in the passive diffusion measuring device 750 (FIG. 25A), and raw skin was used for the permeable membrane 764. Each respective active agent layer 16 included glycerol (approximately 10 wt %), HPC (approximately 0.5 wt %) Procaterol-HCl (approximately 2.5 wt %), and a buffer solution. Two different pH buffer solutions were used. FIG. 31 shows the amount of active agent delivered to the reservoir 772, which has PBS fluid 774 therein, versus time for six delivery devices, individually referenced as devices 501-506. Table 20A shows flux rate measured for the test devices 501, 502, and 503, which used a pH 4.0 buffer solution. The flux rates were calculated using data taken at 8.0 hours. Table 20B shows flux rate measured for the test devices 504, 505, and 506, which used a pH 5.0 buffer solution. The flux rates were calculated using data taken at 8.0 hours. Eight further test devices from the one lot, individually referenced as test devices 507-514, were analyzed to determine the amount of active agent present in each device. Table 20C shows active agent amount and concentration details for the delivery devices 507-510, which used the pH 4.0 buffer solution. Table 20D shows active agent amount and concentration details for the delivery devices 511-514, which used the pH buffer 5.0 solution.

TABLE 20A

| | pH of Buffer | Flux rate at 8.0 hr |
|---|---|---|
| | Solution | (μg/hr/cm²) |
| Delivery Device 501 | 4.0 | 0.20 |
| Delivery Device 502 | 4.0 | 0.17 |
| Delivery Device 503 | 4.0 | 0.13 |
| Ave. | | 0.17 |
| S.D. | | 0.03 |

TABLE 20B

| | pH of Buffer | Flux rate at 8.0 hr |
|---|---|---|
| | Solution | (μg/hr/cm²) |
| Delivery Device 504 | 5.0 | 0.18 |
| Delivery Device 505 | 5.0 | 0.59 |
| Delivery Device 506 | 5.0 | 0.54 |
| Ave. | | 0.44 |
| S.D. | | 0.22 |

TABLE 20C

| | pH of Buffer | Amount Of | Density of Active |
|---|---|---|---|
| | Solution | Active Agent (μg) | Agent (μg/cm²) |
| Delivery Device 507 | 4.0 | 20.17 | 17.85 |
| Delivery Device 508 | 4.0 | 19.80 | 17.52 |
| Delivery Device 509 | 4.0 | 19.22 | 17.01 |
| Delivery Device 510 | 4.0 | 21.33 | 18.88 |
| Ave. | | 20.13 | 17.81 |
| S.D. | | 0.89 | 0.79 |

TABLE 20D

| | pH of Buffer | Amount Of | Density of Active |
|---|---|---|---|
| | Solution | Active Agent (μg) | Agent (μg/cm²) |
| Delivery Device 511 | 5.0 | 20.65 | 18.27 |
| Delivery Device 512 | 5.0 | 22.93 | 20.29 |
| Delivery Device 513 | 5.0 | 21.58 | 19.10 |
| Delivery Device 514 | 5.0 | 21.81 | 19.30 |
| Ave. | | 21.74 | 19.24 |
| S.D. | | 0.94 | 0.83 |

### Example 7

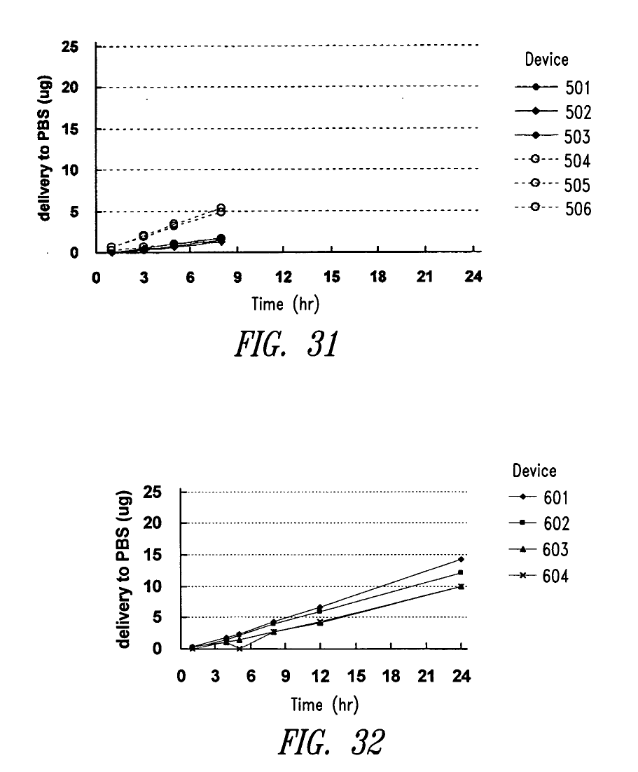

In Example 7, eight delivery devices were prepared according to the embodiment shown in FIGS. 1-2B. The surface area for each respective active agent layer 16 was approximately 1.12 cm². In Example 7, the delivery devices were tested in a Franz cell, and raw skin was used for a permeable membrane. Each respective active agent layer 16 included glycerol (approximately 10 wt %), HPC (approximately 0.5 wt %), and Procaterol-HCl (approximately 2.5 wt %). FIG. 32 shows the amount of active agent delivered to the reservoir 772, which has PBS fluid 774 therein, versus time for four delivery devices, individually referenced as devices 601-604. Table 21A shows flux rate measured for the test devices 601-604, calculated using data taken at 12.0 hours. Four further test devices from the one lot, individually referenced as test devices 605-608, were analyzed to determine the amount of active agent present in each device. Table 21B shows active agent amount and concentration details for the delivery devices 605-608.

TABLE 21A

| | Flux rate at |
|---|---|
| | 12.0 hr |
| | (μg/hr/cm²) |
| Delivery Device 601 | 0.50 |
| Delivery Device 602 | 0.45 |
| Delivery Device 603 | 0.31 |
| Delivery Device 604 | 0.32 |
| Ave. | 0.39 |
| S.D. | 0.09 |

TABLE 21B

| | Amount Of Active Agent | Density of Active Agent |
|---|---|---|
| | (μg) | (μg/cm²) |
| Delivery Device 605 | 24.82 | 21.96 |
| Delivery Device 606 | 22.11 | 19.56 |
| Delivery Device 607 | 24.03 | 21.26 |
| Delivery Device 608 | 22.11 | 19.56 |
| Ave. | 23.50 | 20.79 |
| S.D. | 1.18 | 1.04 |

What is claimed is:

1. A passive transdermal delivery device comprising:
a backing substrate; and
an active agent layer, wherein the active agent layer is substantially anhydrous and oil-free and includes a thickening agent and an ionizable active agent, and wherein the ionizable active agent is electrically neutral in the active agent layer and dissociates into an ionized active agent upon contacting an aqueous medium.

2. The passive transdermal delivery device of claim 1 wherein the ionizable active agent is a salt of an amine-containing active agent.

3. The passive transdermal delivery device of claim 2 further comprising a humectant.

4. The passive transdermal delivery device of claim 2 wherein the thickening agent is HPC, the ionizable active agent is Procaterol HCl and the humectant is urea.

5. The passive transdermal delivery device of claim 2 wherein the ionizable active agent is a β-adrenergic agonist.

6. The passive transdermal delivery device of claim 3 wherein the β-adrenergic agonist is Procaterol HCl.

7. The passive transdermal delivery device of claim 2 wherein the ionizable active agent is a “caine” class analgesic or anesthetic.

8. The passive transdermal delivery device of claim 7 wherein the ionizable active agent is Lidocaine HCl.

9. The passive transdermal delivery device of claim 1 wherein the ionizable active agent is a salt of a carboxylic acid-containing active agent.

10. The passive transdermal delivery device of claim 9 wherein the ionizable active agent is alkaline Diclofenac.

11. The passive transdermal delivery device of claim 1 wherein the ionizable active agent is L-ascorbic acid or a derivative thereof.

12. The passive transdermal delivery device of claim 11 wherein the ionizable active agent is ascorbic acid 2-glucoside.

13. The passive transdermal delivery device of claim 1 wherein the thickening agent is a cellulose derivative.

14. The passive transdermal delivery device of claim 13 wherein the thickening agent is hydroxypropyl cellulose, hydroxymethyl cellulose, hydroxypropyl methylcellulose, or a combination thereof.

15. The passive transdermal delivery device of claim 14 further comprising one or more humectants selected from urea, glycerine, propylene glycol, glyceryl triacetate, and polyols.

16. The passive transdermal delivery device of claim 1 wherein at least 50% of an initial amount of the ionizable active agent is permeable through skin.

17. The passive transdermal delivery device of claim 16 wherein the ionizable active agent is Procaterol HCl.

18. The passive transdermal delivery device of claim 1 further comprising an ionizable additive.

19. The passive transdermal delivery device of claim 18 wherein the ionizable active agent is an alkaline Diclofenac and the ionizable additive is potassium chloride.

20. The passive transdermal delivery device of claim 1 further comprising a replenish layer including additional ionizable active agent and an ion exchange material.

21. A topical formulation comprising:
a thickening agent,
an ionized active agent; and
an aqueous medium, wherein the topical formulation is substantially oil-free.

22. The topical formulation of claim 21 wherein the thickening agent is a cellulose derivative.

23. The topical formulation of claim 21 wherein the ionized active agent is cationic Procaterol cation, anionic Diclofenac, cationic Lidocaine or anionic AA2G.

24. A method of treating a condition associated with an obstructive respiratory ailment in a subject comprising:
applying to the subject's skin a passive transdermal delivery device comprising: a backing substrate; and an active agent layer, wherein the active agent layer is substantially anhydrous and oil-free and includes a thickening agent and an ionizable active agent, and wherein the ionizable active agent is electrically neutral in the active agent layer and dissociates into an ionized active agent upon contacting an aqueous medium; and
allowing the ionizable active agent to dissociate into the ionized active agent.

25. The method of claim 24 comprising contacting the ionizable active agent to sweat of the subject's skin to produce the ionized active agent.

26. The method of claim 25 wherein the ionizable active agent is Procaterol HCl.

27. The method of claim 25 wherein the active agent layer further comprises a humectant.

28. The method of claim 24 wherein the active agent layer comprises HPC, Procaterol HCl and urea.

29. The method of claim 24 wherein at least 50% of the Procaterol HCl is delivered through the skin of the subject within a 24 hour period.
